(19) United States
(12) Patent Application Publication
(10) Pub. No.: US 2013/0288323 A1
(43) Pub. Date: Oct. 31, 2013

(54) MICROBIAL PRODUCTION OF POLYHYDROXYALKANOATES

(71) Applicants: David B. Levin, Winnipeg (CA); Parveen Sharma, Winnipeg (CA)

(72) Inventors: David B. Levin, Winnipeg (CA); Parveen Sharma, Winnipeg (CA)

(73) Assignee: UNIVERSITY OF MANITOBA, Winnipge (CA)

(21) Appl. No.: 13/991,383

(22) PCT Filed: Nov. 29, 2011
(86) PCT No.: PCT/CA11/01322

§ 371 Date: Jul. 16, 2013

Related U.S. Application Data
(60) Provisional application No. 61/418,823, filed on Dec. 1, 2010.

(51) Int. Cl. C12P 7/42

(52) U.S. Cl. CPC C12P 7/42
USPC 435/146; 435/253.3; 435/252.34

(57) ABSTRACT

A process for producing medium-chain-length 3-hydroxyalkanoic acids in a liquid medium, the process comprising: (i) intermixing a liquid culture medium and a microbial inoculum comprising a pure culture of a Pseudomonas putida strain having a portion of a phaC1 gene deleted and/or a portion of a phaC2 gene deleted and/or a portion of a phaZ gene deleted, (ii) culturing the mixture for a period of time to allow for microbial cell growth to occur in the liquid culture medium thereby producing a spent liquid medium and a plurality of microbial cells wherein medium-chain-length 3-hydroxyalkanoic acids are synthesized, (iii) separating the medium-chain-length 3-hydroxyalkanoic acids from the plurality of microbial cells and the spent liquid medium; and (iv) recovering the separated medium-chain-length 3-hydroxyalkanoic acids. A pure culture comprising transgenic microbial cells wherein a portion of a phaC1 gene and/or a phaC2 gene and/or a phaZ gene has been deleted.

### TECHNICAL FIELD

Various embodiments disclosed herein generally relate to production of biodegradable polyhydroxyalkanoates. Some embodiments relate to biological processes for production of biodegradable polyhydroxyalkanoates. Some embodiments relate to novel microorganisms for use in the biological processes for production of biodegradable polyhydroxyalkanoates.

### BACKGROUND

Polyhydroxyalkanoates (PHAs) have attracted considerable attention as source materials for production of biodegradable plastics as alternatives for petroleum-based plastics. Due to their inherent biocompatibility and biodegradability properties, PHAs are receiving considerable commercial interest as replacements for petroleum-derived plastics which are typically are recalcitrant in the environment and persist for extremely long periods of time after they are discarded into landfills. Furthermore, petroleum-derived plastics are typically produced from non-renewable resources, while PHAs can be produced from renewable sources such as sugars and oils derived from plants.

A wide variety of microorganisms synthesize and accumulate PHAs as carbon and energy storage materials. The monomeric composition of PHAs depends both on the type bacterial strain(s) used and on the type of carbon source(s) supplied. Short chain-length PHAs (scl-PHAs) contain 3-hydroxy monomers with chain lengths of C4 and C5, while medium chain-length PHAs (mcl-PHAs) contain 3-hydroxy monomers with chain lengths from C6 to C14. scl-PHAs and mcl-PHAs are synthesized by different metabolic pathways and a single selected microbial culture will typically synthesize one type of PHAs but not both types.

Currently commercially available PHAs are the scl-PHAs which generally comprise copolymers of 3-hydroxybutyrate (HB) and hydroxyvalerate (HV), homopolymers of HB, copolymers of HB and hydroxyhexanoate (HHx). These polymers are generally produced by pure cultures of naturally occurring microorganisms such as Alcaligenes latus and Burkholderia sacchari, or by genetically modified microbial strains, such as recombinant Escherichia coli or Wautersia eutropha. PHAs with different ratios of monomers are produced by different bacteria and have different physical properties. Microbial production of scl-PHAs has been extensively studied and there are numerous efficient commercial production systems in use. Consequently, scl-PHAs are readily available and are relatively inexpensive.

scl-PHAs are highly crystalline, rigid, and brittle with poor elastic properties that limit their potential for commercial applications. mcl-PHAs are semi-crystalline elastomers with low melting points, low tensile strength, and high elongation/break ratios. These features make mcl-PHAs preferable feedstocks for production of biodegradable plastics because they are more ductile and easier to mold than are scl-PHAs. It is known that Pseudomonads belonging to the rRNA homology group I, such as Pseudomonas fluorescens and Pseudomonas putida, are able to synthesize mcl-PHAs. However, mcl-PHAs are difficult to produce in large-volume production systems using these microorganisms. As a result, mcl-PHAs are currently only available in limited volumes and are very expensive.

### SUMMARY OF THE INVENTION

This section provides a general summary of the disclosure and is not a comprehensive disclosure of its full scope.

Disclosed herein is a process for producing medium-chain-length 3-hydroxyalkanoic acids in a liquid medium inoculated with a pure culture of a Pseudomonas putida strain selected from the group consisting of, IDAC 181110-01, IDAC 181110-02, IDAC 181110-03, STRAIN C1-2, IDAC 171111-01, IDAC 171111-01, and combinations thereof. The liquid culture medium may be a carbohydrate-rich and/or oil-rich agri-industrial waste stream. The liquid culture medium may be supplemented with one or more of 5-carbon monosaccharides and/or one or more 6-carbon monosaccharides and/or one or more disaccharides and/or one or more plant oil and/or saturated fatty acids. The supplemented or un-supplemented liquid culture medium may be supplemented with salicylic acid.

Disclosed herein are novel naturally occurring strains of Pseudomonas sp. isolated from sludge produced in waste water treatment plants and from animal sewage washings, using minimal culture media supplemented with a carbohydrate-rich agri-industrial wastestream, that are particularly efficient producers of large quantities of medium-chain-length 3-hydroxyalkanoic acids.

Also disclosed herein are novel deletion-mutants of selected Pseudomonas putida strains that are particularly efficient producers of large quantities of medium-chain-length 3-hydroxyalkanoic acids. The deletion mutant strains are produced by deleting a portion of the phaC1 and/or phaC2 and/or phaZ genes from the genome of the selected P. putida strain.

### BRIEF DESCRIPTION OF THE FIGURES

The drawings described herein are for illustrative purposes only of selected embodiments and are not intended to limit the scope of the present disclosure.

FIGS. 1(A) and 1(B) are micrographs of purified bacterial strains isolated from hog sewage sludge and cultured on (A) LB agar supplemented with Nile Red dye, and (b) agar containing thin stillage from an ethanol production system supplemented with Nile Red dye. The arrows point to microbial colonies that fluoresced under UV light illumination;

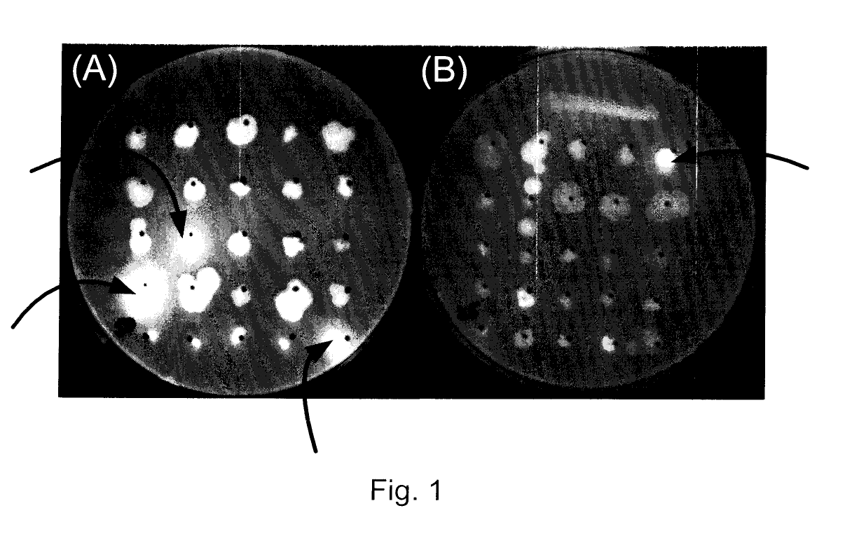

FIGS. 2(A) and 2(B) are micrographs of Pseudomonas putida strain LS46 cultured on supplemented with glucose and Nile Red dye wherein (A) was taken under bright-field microscopy and (B) was taken under fluorescence microscopy;

FIGS. 3(A) and 3(B) are micrographs of P. putida strain LS5 cultured on supplemented with glucose and Nile Red dye wherein (A) was taken under bright-field microscopy and (B) was taken under fluorescence microscopy;

FIG. 4 is a chart showing the results of GC analysis of PHA methyl esters produced by P. putida strain LS46;

FIGS. 5(A)-5(C) are charts showing identification using GC-MS anysis of a PHA produced by P. putida strain LS46 wherein (A) is a mass spectra of compounds recovered from culture media, (B) shows the best match of the PHA with the NIS library, and (C) shows the identified compound 3-hydroxydecanoate;

FIG. 6 is a chart showing the production of cell dry weight and PHAs by P. putida strain LS46 on LB medium (LB-CDW and LB-PHA respectively), and on LB medium supplemented with 2 g/L glucose (LBG-CDW and LBG-PHA respectively);

FIGS. 7(A) and 7(B) are charts showing the production of cell dry weight and PHAs by P. putida strain LS46 in (A) culture medium with limited nitrogen availability, and (b)—limiting culture medium provided with excess nitrogen;

FIGS. 8(A) and 8(B) are charts showing the results of neighbor-tree analyses of Pseudomonas sp. on the basis of their homologies of (A) the recA gene, and (B) the groEL gene;

FIG. 9 is a schematic diagram showing the organization of pha synthesis genes in P. putida strain LS46;

FIG. 10 is a schematic flow chart illustrating an exemplary strategy for deleting pha genes from the genome of P. putida;

FIG. 11 is a micrograph of polyacrylamide gels confirming a 3.8 kb deletion in the pha region of the pJQ200Δpha plasmid;

FIG. 12 is a is a micrograph of polyacrylamide gels confirming deletion of the phaZ from the genome of region of P. putida strain LS46 (lane 1 is the pJQ200ΔZ plasmid; lane 2 is the P. putida deletion mutant stain Z502; lane 3 is the parent P. putida strain LS46; lane 4 is a reference 1 kb ladder);

FIG. 13 is a chart comparing the production of PHAs in Ramsey's medium enriched with 2% glusose, of the parent P. putida strain LS46 and the phaZ deletion mutant P. putida strain Z502;

FIGS. 14(A) and 14(B) are charts showing production of different PHA monomers in Ramsey's medium enriched with 2% glusose by (A) the parent P. putida strain LS46, and (B) the phaZ deletion mutant P. putida strain Z502;

FIG. 15 is a chart comparing the production of PHAs in Ramsey's medium enriched with 20 mM octanoic acid, of the parent P. putida strain LS46 and the phaZ deletion mutant P. putida strain Z502; and

FIG. 16 is a chart comparing the production of 3-hydroxyoctanoate in Ramsey's medium enriched with 20 mM octanoic acid, of the parent P. putida strain LS46 and the phaZ deletion mutant P. putida strain Z502.

### DETAILED DESCRIPTION OF THE INVENTION

The present disclosure relates to novel bacterial strains useful for production of mcl-PHAs, and to processes for production of mcl-PHAs using the novel bacterial strains.

Some embodiments of the present invention relate to methods for isolation, recovery and purification of novel bacterial strains that have the metabolic capability to produce large volumes of mcl-PHAs in liquid production systems. Some embodiments relate to methods of genetically engineering novel bacterial strains that have metabolic capability to produce large volumes of mcl-PHAs in liquid production systems.

Some embodiments of the present invention relate to methods for production of mcl-PHAs using novel bacterial strains produced as disclosed herein.

Some embodiments of the present invention relate to novel bacterial strains useful for production of mcl-PHAs. Other embodiments of the present invention relate to consortia of bacterial strains for production of mcl-PHAs in liquid systems.

Unless otherwise defined, all technical and scientific terms used herein have the same meaning as commonly understood by one of ordinary skill in the art to which this invention belongs. In order that the invention herein described may be fully understood, the following terms and definitions are provided herein.

The word “comprise” or variations such as “comprises” or “comprising” will be understood to imply the inclusion of a stated integer or groups of integers but not the exclusion of any other integer or group of integers.

The term “hydroxy acid” as used herein means a carboxylic acid in which one or more hydrogen atoms of the aliphatic or aromatic group has been replaced by a hydroxyl group.

The term “polyhydroxy acids” as used herein means a polymer of repeating hydroxyl acid monomer units.

The term “polyhydroxalkanoates”, also referred to herein as “PHAs”, are linear polyesters produced by bacterial fermentation of sugars and/or lipids. PHAs are generally formed within bacterial cells as refractive granules comprising: (i) homopolyesters with the same hydroxyalkanoic acids, or (ii) copolyesters with different hydroxyalkanoic acids. PHAs are useful for production of biodegradable bioplastics by combining selected PHAs with other polymers, enzymes and inorganic materials.

The term “polyester” refers to the category of polymers that contains a functional ester group as their main chain.

The term “short-chain-length polyhydroxalkanoates”, also referred to herein as “scl-PHAs” are poly(3-hydroxyalkanoates) classified as having 3 to 5 carbons in their repeating units.

The term “medium-chain-length polyhydroxalkanoates”, also referred to herein as “mcl-PHAs” are poly(3-hydroxyalkanoates) classified as having more than 5 carbons in their repeating units.

The term “C5” is used herein for convenience to represent a polyhydroxyalkanoate monomer that has five carbons (three of the carbons in its backbone and two carbons in its sidechain) and is named polyhydroxypentanoate.

The term “C6” is used herein for convenience to represent a polyhydroxyalkanoate monomer that has six carbons (three of the carbons in its backbone and three carbons in its sidechain) and is named polyhydroxyhexanoate.

The term “C8” is used herein for convenience to represent a polyhydroxyalkanoate monomer that has eight carbons (three of the carbons in its backbone and five carbons in its sidechain) and is named polyhydroxyoctanoate.

The term “C10” is used herein for convenience to represent a polyhydroxyalkanoate monomer that has ten carbons (three of the carbons in its backbone and seven carbons in its sidechain) and is named polyhydroxydecanoate.

The term “C12” is used herein for convenience to represent a polyhydroxyalkanoate monomer that has twelve carbons (three of the carbons in its backbone and nine carbons in its sidechain) and is named polyhydroxydodecanoate.

The term “C14” is used herein for convenience to represent a polyhydroxyalkanoate monomer that has six carbons (three of the carbons in its backbone and eleven carbons in its sidechain) and is named polyhydroxytetradecanoate.

The term “a cell” includes a single cell as well as a plurality or population of cells.

The term “consortium” as used herein means a microbial inoculum comprising a combination of two or more microbial strains

The term “about” or “approximately” means within 20%, preferably within 10%, and more preferably within 5% of a given value or range.

The term “nucleic acid” refers to a polymeric compound comprised of covalently linked subunits called nucleotides. Nucleic acid includes ribonucleic acid (RNA) and deoxyribonucleic acid (DNA), both of which may be single-stranded or double-stranded. DNA includes cDNA, genomic DNA, synthetic DNA, and semi-synthetic DNA.

The term “gene” refers to an assembly of nucleotides that encode a polypeptide, and includes cDNA and genomic DNA nucleic acids.

The term “recombinant DNA molecule” refers to a DNA molecule that has undergone a molecular biological manipulation.

The term “vector” refers to any means for the transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes plasmids, liposomes, electrically charged lipids (cytofectins), DNA-protein complexes, and biopolymers. In addition to a nucleic acid, a vector may also contain one or more regulatory regions, and/or selectable markers useful in selecting, measuring, and monitoring nucleic acid transfer results (transfer to which tissues, duration of expression, etc.).

The term “cloning vector” refers to a replicon, such as plasmid, phage or cosmid, to which another DNA segment may be attached so as to bring about the replication of the attached segment. Cloning vectors may be capable of replication in one cell type, and expression in another (“shuttle vector”).

A cell has been “transfected” by exogenous or heterologous DNA when such DNA has been introduced inside the cell. A cell has been “transformed” by exogenous or heterologous DNA when the transfected DNA effects a phenotypic change. The transforming DNA can be integrated (covalently linked) into chromosomal DNA making up the genome of the cell.

The term “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms.

Modification of a genetic and/or chemical nature is understood to mean any mutation, substitution, deletion, addition and/or modification of one or more residues. Such derivatives may be generated for various purposes, such as in particular that of enhancing its production levels, that of increasing and/or modifying its activity, or that of conferring new biological properties on it. Among the derivatives resulting from an addition, there may be mentioned, for example, the chimeric nucleic acid sequences comprising an additional heterologous part linked to one end, for example of the hybrid construct type consisting of a cDNA with which one or more introns would be associated.

Likewise, for the purposes of the invention, the claimed nucleic acids may comprise promoter, activating or regulatory sequences, and the like.

The term “promoter sequence” refers to a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

The term “homologous” in all its grammatical forms and spelling variations refers to the relationship between proteins that possess a “common evolutionary origin,” including homologous proteins from different species. Such proteins (and their encoding genes) have sequence homology, as reflected by their high degree of sequence similarity. This homology is greater than about 75%, greater than about 80%, greater than about 85%. In some cases the homology will be greater than about 90% to 95% or 98%.

“Amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence.

The term “polypeptide” refers to a polymeric compound comprised of covalently linked amino acid residues. Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. A polypeptide of the invention preferably comprises at least about 14 amino acids.

The term “protein” refers to a polypeptide which plays a structural or functional role in a living cell.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

The term “derivative” refers to a product comprising, for example, modifications at the level of the primary structure, such as deletions of one or more residues, substitutions of one or more residues, and/or modifications at the level of one or more residues. The number of residues affected by the modifications may be, for example, from 1, 2 or 3 to 10, 20, or 30 residues. The term derivative also comprises the molecules comprising additional internal or terminal parts, of a peptide nature or otherwise. They may be in particular active parts, markers, amino acids, such as methionine at position −1. The term derivative also comprises the molecules comprising modifications at the level of the tertiary structure (N-terminal end, and the like). The term derivative also comprises sequences homologous to the sequence considered, derived from other cellular sources, and in particular from cells of human origin, or from other organisms, and possessing activity of the same type or of substantially similar type. Such homologous sequences may be obtained by hybridization experiments. The hybridizations may be performed based on nucleic acid libraries, using, as probe, the native sequence or a fragment thereof, under conventional stringency conditions or preferably under high stringency conditions.

We have surprisingly discovered certain naturally occurring strains of P. putida that were isolated and recovered from municipal sewage sludge and from animal waste removal and washing systems, are particularly efficient producers of mcl-PHAs. Accordingly, some embodiments of the present invention relate to methods for isolating and recovering naturally occurring microbial strains that are efficient producers of mcl-PHAs.

Some embodiments of the present invention relate to genetically modifying naturally occurring microbial strains to improve their ability to efficiently produce mcl-PHAs. We have discovered that deletions made in one or more of the phaC1 gene, the phaC2 gene, and the phaZ gene results in significant increases in mcl-PHAs production when compared to mcl-PHAs production by the wild-type parent microbial strains.

Some embodiments of the present invention relate to processes for the use of industrial waste streams and effluents as feedstocks for the production of mcl-PHAs by the naturally occurring microbial strains and/or geneticially modified microbial strains disclosed herein. Industrial waste streams suitable for use in the mcl-PHAs production processes are exemplified by carbohydrate-rich and/or oil-rich agri-industrial waste streams such as cellulose fermentation products generated by cellulolytic bacteria, thin stillage and slurries recovered from distillation of ethanol in grain ethanol production systems, thin stillage and slurries recovered from distillation of ethanol in cellulosic ethanol production systems, hydrolysates produced by enzymatic or chemical digestion of hemicelluloses. The industrial waste streams my be thin fluids, viscous fluids, and may contain particulates and/or semisolids materials and/or solids materials. Also suitable as feedstocks for the processes of the present invention for production of mcl-PHAs are “dried distiller grains with solubles” (DDGS). DDGS are major by-products of ethanol production from grains such as corn and wheat, and are commonly used as a feed supplement in the livestock industry. DDGS contains 9.6% fibre, 32% protein, 12% fat, and 2-3% solubles. DDGS are manufactured from whole stillage, the spent biomass from ethanol production. Whole stillage is subjected to centrifugation to separate the soluble fraction, called thin stillage, from the suspended solids, which are concentrated into a wet cake. The thin stillage is further concentrated by evaporation and the syrup derived from this is mixed with the wet cake, and then dried to form the DDGS. The thin stillage and wet cake contain different amounts and types of nutrients that make these fermentation products also suitable as feedstocks for microbial production of mcl-PHAs.

It is within the scope of the present invention to supplement industrial waste stream feedstocks for production of mcl-PHAs with processes disclosed herein, with one or more 5-carbon monosaccharides and/or one or more 6-carbon monosaccharides and/or one or more disaccharides and/or one or more plant oils.

Suitable 5-carbon monosaccharides are exemplified by arabinose, lyxose, ribose, xylose, ribulose, and xylulose. The 5-carbon monosaccharides can be added into an industrial waste stream feedstock at a concentration of about 0.5% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w).

Suitable 6-carbon monosaccharides are exemplified by glucose, fructose, mannose, galactose, allose, altose, idose, and talose. The 6-carbon monosaccharides can be added into an industrial waste stream feedstock at a concentration of about 0.5% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w).

Suitable disaccharides are exemplified by sucrose, lactose, lactulose, maltose, and trehalose. The disaccharides can be added into an industrial waste stream feedstock at a concentration of about 0.5% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w).

Suitable plant oils are exemplified by artichoke oil, canola oil, coconut oil, corn oil, cottonseed oil, mustard oil, olive oil, palm oil, peanut oil, rapeseed oil, safflower oil, soybean oil, sunflower oil. The plant oils can be added into an industrial waste stream feedstock at a concentration of about 0.5% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w).

It is within the scope of the present invention to supplement a industrial waste stream feedstock for production of mcl-PHAs with processes disclosed herein, with one or more saturated fatty acids and/or salicylic acid.

Suitable saturated fatty acids are exemplified by octanoic acid, decanoic acid, dodecanoic acid, tetradecanoic acid, salts thereof, esters thereof. The saturated fatty acids can be added into the industrial waste stream feedstock at a concentration of about 0.5 mM, 1.0 mM, 2.0 mM, 3.0 mM, 4.0 mM, 5.0 mM, 10 mM, 15 mM, 20 mM, 25 mM, 30 mM, 35 mM, 40 mM, 45 mM, 50 mM, 55 mM, 60 mM, 65 mM, 70 mM.

Salicylic acid can be added into the industrial waste stream feedstock at a concentration of about 0.5 mM, 1.0 mM, 1.5 mM, 2.0 mM, 2.5 mM, 3.0 mM, 3.4 mM, 4.0 mM, 4.5 mM, 5.0 mM, 5.5 mM, 6.0 mM, 7.0 mM, 7.5 mM.

An exemplary method for isolating and recovering naturally occurring microbial strains useful for production of mcl-PHAs in liquid cultures. First a sample of a sewage material is collected from a suitable source. Suitable sewage material is exemplified by sludge from municipal waste treatment facilities, sewage materials such as liquid and semi-solid manures and/or floor washings from livestock production facilities. Serial dilutions are prepared from the sewage material sample are plated onto a nutritionally enriched agar medium and then cultured for a period of time required for microbial cultures to grow on the surface of the agar plates. Suitable nutritionally enriched agar media are exemplified by Luria-Bertani medium, tryptic-soy medium, Ham's nutrient medium among others. Suitable temperatures for culturing the dilution plates are from the range of about 15° C. to about 37° C., about 15° C. to about 37° C., about 20° C. to about 37° C., about 25° C. to about 37° C., about 30° C. to about 37° C. Single colonies are picked from the dilution plates, streaked onto fresh agar medium comprising an ethanol fermentation by-product as the sole carbon source. A suitable ethanol fermentation by-product is exemplified by cellulose fermentation products generated by cellulytic bacteria, thin stillage and slurries recovered from distillation of ethanol in grain ethanol production systems, thin stillage and slurries recovered from distillation of ethanol in cellulosic ethanol production systems, hydrolysates produced by enzymatic or chemical digestion of hemicelluloses, DDGS, wet cake, and mixtures thereof. The streaked microbial plates are cultured until microbial colonies are established after which, individual colonies are picked, streaked onto fresh agar medium comprising an ethanol fermentation by-product as the sole carbon source and cultured again until microbial colonies are established. The process is repeated until pure cultures are established. The microbial isolates are tested for their capability to produce mcl-PHAs by culturing on an enriched nutrient medium supplemented with Nile red dye, and then assessed for the fluorescence under UV illumination. Fluorescing isolates are then assessed for the presence with their microbial cells of granules that fluoresce under UV illumination; The presence of fluorescing granules indicates the presence of mcl-PHAs.

An exemplary embodiment of the present invention relates to a process for production of mcl-PHAs by one or more of the naturally occurring microbial isolates isolated and recovered as disclosed herein, using a liquid feedstock comprising a carbohydrate-rich and/or oil-rich agri-industrial waste stream. An agri-industrial waste stream suitable for use as a liquid feedstock for the process is exemplified by fermentation products generated by cellulytic bacteria, thin stillage and slurries recovered from distillation of ethanol in grain ethanol production systems, thin stillage and slurries recovered from distillation of ethanol in cellulosic ethanol production systems, hydrolysates produced by enzymatic or chemical digestion of hemicelluloses. The industrial waste streams my be thin fluids, viscous fluids, and may contain particulates and/or semisolids materials and/or solids materials. It is preferable that the carbon content of the agri-industrial waste stream is greater than 1% (w/w). The carbon content of the agri-industrial waste stream may be in the range of about 1% to about 30%, about 2% to about 20%, about 2% to about 10%. It is preferable that the availability of nitrogen in the agri-industrial waste stream is limited. The nitrogen content of the agri-industrial waste stream is preferably less than 4%, less than 3%, less than 2%, less than 1%.

The liquid feedstock may be supplemented with one or more 5-carbon monosaccharides and/or one or more 6-carbon monosaccharides and/or one or more disaccharides and/or one or more plant oils. The liquid feedstock may be supplemented with a saturated fatty acid and/or a salt of a saturated fatty acid and/or an ester of a saturated fatty acid. The liquid feedstock may be supplemented with salicylic acid.

The present process for production of mcl-PHAs is performed in a vessel suitable for culturing therein a microbial inoculum in the liquid feedstock. The microbial inoculum may comprise Psuedomonas putida strain LS1, P. putida strain LS5, P. putida strain LS33, P. putida strain LS34, P. putida strain LS39, P. putida strain LS46, P. putida strain Pc1-5, P. putida strain Z502, or combinations thereof. When a combination of two or more P. putida strains are used as an inoculum, the combination may be referred to as a “consortium”. The microbial culture growing out from the microbial inoculm, is preferably continually mixed during the culture period by agitation and/or aeration and/or stirring and/or turbulating or the like. The process may be a batch production process or alternatively, continuous production process.

A batch production process according to an exemplary embodiment of the present invention comprises providing into a reaction vessel a predetermined volume of a selected liquid feedstock, mixing thereinto an inoculum comprising a selected microbial strain, or alternatively, a consortium of strains produced as disclosed herein, culturing the inoculated liquid medium for a period of time sufficient to provide a maximum production of microbial cells followed by a maximum production of mcl-PHAs resulting in a spent liquid medium, separating the mcl-PHAs from the spent liquid medium and the microbial cells, and then recovering the mcl-PHAs. It is within the scope of the present invention to further process the recovered mcl-PHAs to remove impurities and to optionally separate and recover individual C5 monomers, C6 monomers, C8 monomers, C10 monomers, C12 monomers, and C14 monomers comprising the mcl-PHAs.

A continuous production process according to an exemplary embodiment of the present invention comprises providing a constant feed of a selected liquid medium into a reaction vessel wherein is continuously cultured a selected microbial strain or alternatively a consortium of consortium of strains produced as disclosed herein, egressing from the reaction vessel a volume of liquid medium comprising the microbial culture at a rate equivalent to the rate of constant feed of liquid medium into the reaction vessel, separating the mcl-PHAs from the egressed liquid medium and microbial cells contained therein, and then recovering the mcl-PHAs. It is within the scope of the present invention to further process the recovered mcl-PHAs by to remove impurities and to optionally separate and recover individual types of C5, C6, C8, C10, C12, and C14 monomers comprising the mcl-PHAs.

It is known that microbial biosynthesis of scl-PHAs is primarily controlled by expression of the phaC gene that encode the expression and production of PHA synthase (Steinbuchel et al., 2001, Biochemical and Molecular Basis of Microbial Synthesis of Polyhydroxyalkanoates in Microorganisms, Int. J. Biol. Macromol. 25:3-19). Furthermore, methods are known for transforming selected microbial strains with the phaC gene for the purpose of achieving overproduction of scl-PHAs in microbial fermentation systems (Lee et al., 2001, Production of Microbial Polyester by Fermentation of Recombinant Microorganisms, Adv. Biochem. Engin. Biotechnol. 71:81-123). In contrast to biosynthesis of scl-PHAs, the biosynthesis of mcl-PHAs in Pseuodmonas species is regulated by two gene loci: (i) a PHA synthase cgene locus composed of phaC1, phaC2, phaZ, and phaD genes, and (ii) a granule-associated gene locus composed of phaF and phal genes (Kim et al., 2006, Metabolic Engineering and Characterization of phaC1 and phaC2 Genes from Pseudomonas putida KCTC1639 for Overproduction of Medium-Chain-Length Polyhydroxyalkanoate, Biotechnol. Prog. 22:1541-1546). Furthermore, it appears that biosynthesis of mcl-PHAs is carried out by phaC1 encoding mcl-PHA synthese I and phaC2 encoding mlc-PHA synthase II, and it has been shown that over-expressing the phaC1 gene results in significantly increased production of mcl-PHAs (Kim et al., 2006). It is also known that the phaZ gene encodes a specific mcl-PHA depolymerase (de Eugenio et al., 2007, Biochemical Evidence That phaZ Gene Encodes a Specific Intracellular Medium Chain Length Polyhydroxyalkanoate Depolymerase in Pseudomonas putida KT2442, J. Biol. Chem. 282(7): 4951-4962), and that over-expression of the phaZ gene in combination with expression of a putative long-chain fatty acid transport proteinfadL and an acyl-CoA synthetase (fadD) results in increased production of mcl-PHAs by recombinant P. putida (Yuan et al., Microbial production of medium-chain-length 3-hydroxyalkanoic acids by recombinant Pseudomonas putida KT2442 harboring genes fadL, fadD and phaZ. FEMS Microbiol Lett. 283: 167-175).

We have surprisingly discovered that deleting portions or all of phaC1, phaC2, and/or phaZ genes from the genome of a selected P. putida strain resulted in increased production of mcl-PHAs by P. putida strains so transformed. Moreover, the ratios of the C6 monomers, C8 monomers, C10 monomers, C12 monomers, and C14 monomers comprising the mcl-PHAs produced by the transformed P. putida strains were altered when compared to the combination of the C6 monomer, C8 monomer, C10 monomer, C12 monomer, and C14 monomer comprising the mcl-PHAs produced by the wild-type parent P. putida strain. Accordingly, another exemplary embodiment of the present invention relates to novel transgenic P. putida strains wherein portions or all of phaC1, phaC2, and/or phaZ genes have been deleted from their genomes. Those skilled in these arts having knowledge of well-known molecular biology methods for producing and culturing deletion mutants will understand how to produce deletion-mutants of P. putida strains that have deletions in one or more of their inherent phaC1, phaC2, and/or phaZ genes in reference to the methods disclosed herein. Suitable deletion-mutants of P. putida strains are exemplified by P. putida strain Pc-1-5, P. putida strain Z327, P. putida strain Z502.

It is within the scope of the present invention to use a phaC1-deletion P. putida mutant strain and/or a phaC2-deletion P. putida mutant strain and/or a phaZ-deletion P. putida mutant strain in the processes disclosed herein for production of mcl-PHAs from a liquid feed stock comprising an un-amended carbohydrate-rich and/or oil-rich agri-industrial waste stream, or alternatively, using a liquid feedstock comprising a carbohydrate-rich and/or oil-rich agri-industrial waste stream amended as disclosed herein. If so desired, one or more selected deletion-mutant P. putida strains can be combined with one or more naturally occurring P. putida strains to form an exemplary microbial inoculum of the present invention.

The following examples are provided to more fully describe the invention and are presented for non-limiting illustrative purposes.

### EXAMPLES

### Example 1

### Isolation and Recovery of Mcl-PHAs Producing Microorganisms from Sewage and Sludge

Sewage sludge samples were collected from the Winnipeg Wastewater Treatment Plant (Winnipeg, MB, CA). Hog barn wash samples were collected from a hog farm in Niverville, Manitoba. The samples were stored at 4° C. and were processed as soon as possible.

Fermentation by-products from a commercial grain ethanol plant (Husky Energy, Minnedosa, MB, CA) namely (i) DDGS, (ii) wet cake, and (iii) thin slurry were collected and stored at 4° C.

Three types of liquid media were prepared using the fermentation by-products as the sole carbon sources: (i) medium 1 contained 10 g/L DDGS, (ii) medium 2 contained 10 g/L wet cake, and (iii) medium 3 contained 10 g/L thin slurry. The pH of each medium was adjusted to 7.0, after which the media were autoclaved at 121° C. for 30 min.

50 mL of each type of liquid medium was inoculated with 1-mL sewage sludge sample or a 1-mL hog barn wash sample, and then were incubated on a rotary shaker at 37° C. After 1 week, serial dilutions from each liquid culture were separately plated on to Luria Bertani (LB) agar plates. A total of 45 bacterial isolations were made from DDGS, wet cake, and thin stillage enriched media. Individual colonies were selected from the LB agar plates and were spotted onto one of (i) LB agar plates supplemented with Nile Red dye (0.5 mg/L), and (ii) agar places containing thin slurry as the sole carbon source and supplement with Nile Red dye (0.5 mg/L). The agar plates were incubated at 30° C. for 7 days, and then observed under UV light. Isolated colonies that fluoresced under UV light were selected and cultured for use in subsequent studies. Seven isolates which fluoresced brightly under UV illumination were selected for further studies.

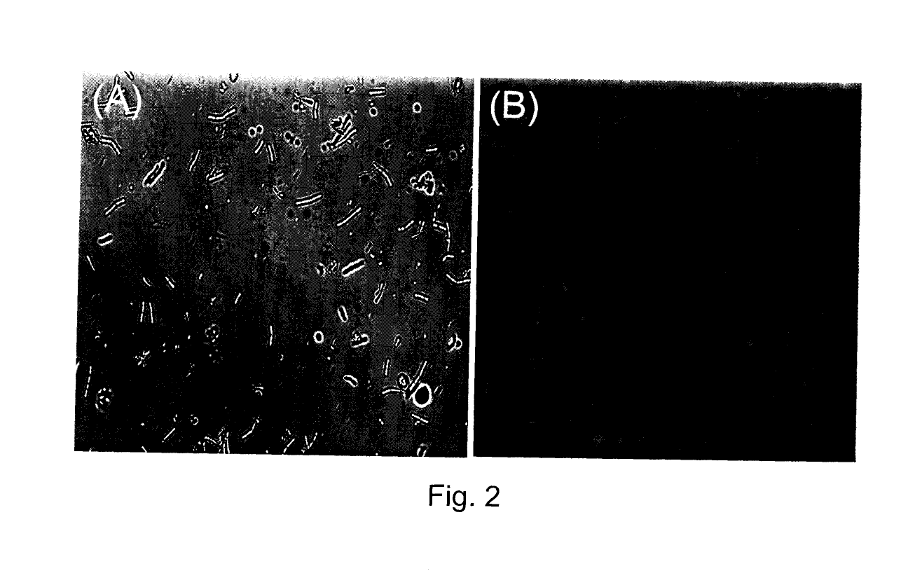

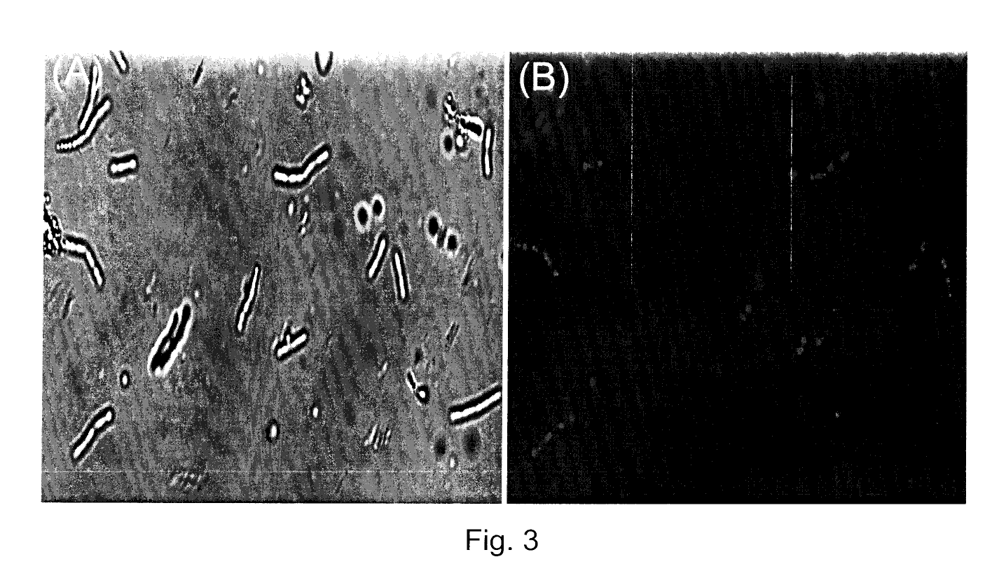

The selected bacterial isolates from the LB agar plates and the thin stillage agar plates were grown at 30° C. in flasks containing (i) liquid LB medium, or (ii) thin stillage liquid medium. After 72 h, 300 μl of bacterial culture from each flask were mixed with 2 drops of 0.2% Nile-blue-solution. The mixtures were vortexed and incubated in a 55° C. water bath at for 10 minutes. Wet mounts of the cultures were then visualized using an oil immersion objective under: (i) bright-field illumination, and (ii) illumination with UV light at an excitation wavelength 543 nm to produce epiflourescence at 598 nm. FIG. 2(A) shows a bright-field microscopy view of a Pseudomonas putida strain LS46 sample while FIG. 2(B) shows the same field under UV illumination. The bright spots in FIG. 2(B) are fluorescent inclusion bodies inside the microbial cells indicating the presence of concentrated PHAs. FIG. 3(A) shows a bright-field microscopy view of a P. putida strain LS5 sample while FIG. 3(B) shows the same field under UV illumination. The bright spots in FIG. 3(B) are fluorescent inclusion bodies inside the microbial cells indicating the presence of concentrated PHAs.

### Example 2

### PHAs Production by the Isolated Microbial Cultures

Six of the microbial isolates (i.e., LS1, LS5, LS33, LS34, LS39, LS46) that produced epifluorescence under UV illumination were separately cultured for 72 h in: (i) 200 mL LB broth, and (ii) 200 mL LB broth supplemented with 3 g/L glucose. The microbial cells were centrifuged at 4500 g for 20 min in a Sorvall RC6-Plus centrifuge. The microbial pellets were washed twice in 0.85% NaCl and then dried at 80° C. for 48 h. The intracellular PHA contents and PHA composition were determined by GC-MS following the method taught by Braunegg et al. (1978, Eur. J. Appl. Microbiol. Biotechnol. 6:29-37). Dried cells (20 mg) were mixed with 1 mL chloroform and 1 mL methanol containing 15% sulphuric acid. Benzoic acid (1 mg/mL) was used as an internal standard. The suspensions were refluxed at 100° C. for 4 h, after which, 0.5 ml of water were added to each suspension. The mixtures were centrifuged at 4000×g. The lower chloroform layers were separated and analyzed using a GC-MS fitted FID detector and aHP-5 capillary column. The initial oven temperature was set at 75° C. for 6 min and then was increased to 265° C. with a ramping rate of 20° C./min.

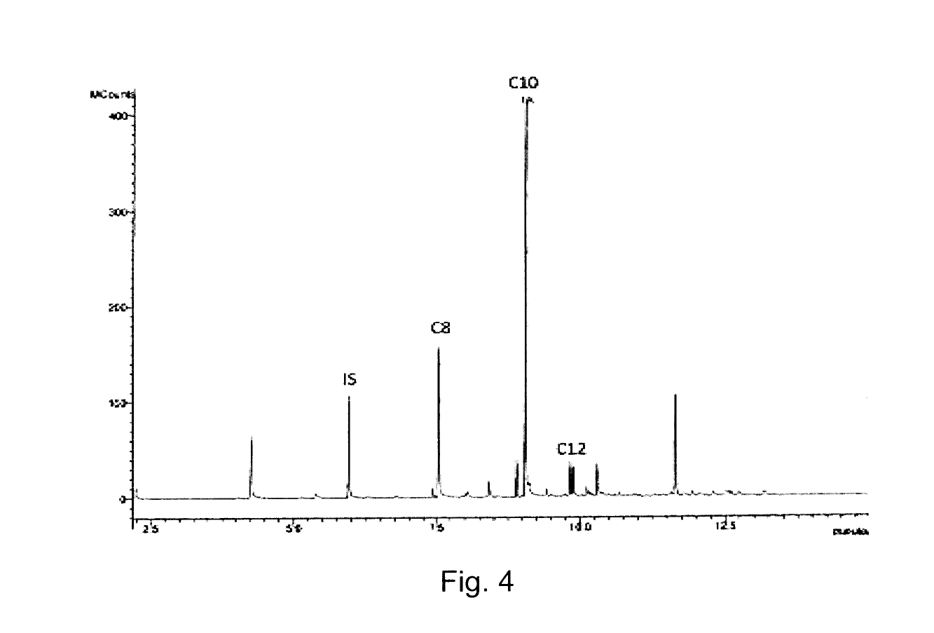

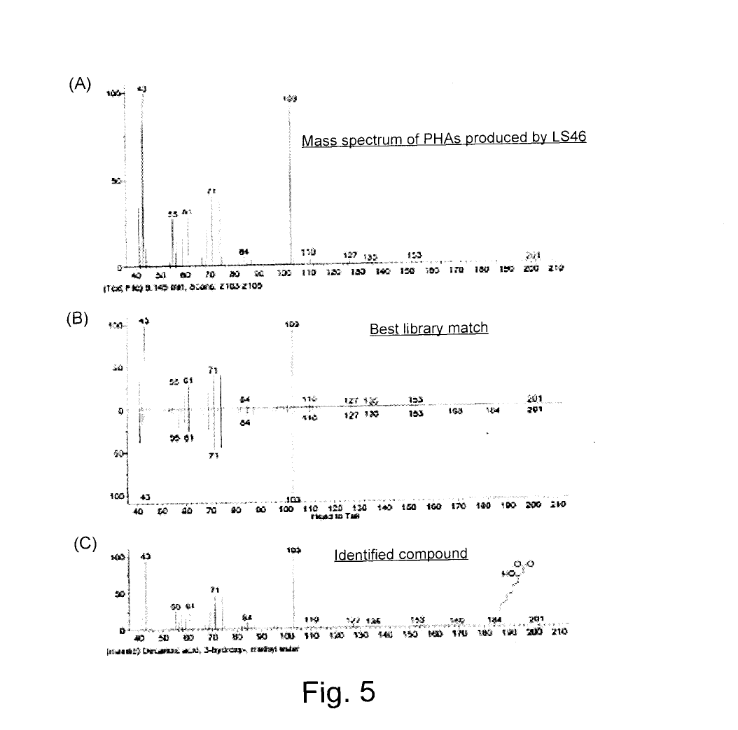

Methyl esters of 3-hydroxyalkanoate monomers produced by the microbial isolates were detected on the basis of their retention times in the GC-MS. FIG. 4 shows the GC-MS results recorded for P. putida strain LS46. 3-hydroxy compounds produced by the 7 microbial isolates were identified by the presence of a signal in their mass spectra at 103 (FIG. 5(A)). The mass spectra was compared to a library of know spectra (FIG. 5(B)) which enabled identification of individual 3-hydroxyalkanoate monomers (FIG. 5(C)).

Table 1 shows the different 3-hydroxyalkanoate monomers produced by the microbial isolates. Isolate LS1 produced 3-hydroxyoctanoate (C8 3-hydroxyalkanoate), 3-hydroxydecanoate (C10 3-hydroxyalkanoate), 3-hydroxydodecanoate (C12 3-hydroxyalkanoate), 3-hydroxytetradecanoate (C14 3-hydroxyalkanoate) and 3-hydroxyoctadecanoate (C18 3-hydroxyalkanoate). The 3-hydroxydodecanoate and 3-hydroxy tetradecanoate were methylated. Isolate LS39 synthesized similar types of monomers as isolate LS1. Isolate LS5 did not produce C18 3-hydroxy acid but produced 3-hydroxyhexanoate (C6 3-hydroxyalkanoate). Isolate LS33 synthesized 3-hydroxydecanoate only. LS34 synthesized 3-hydroxyoctanoate, 3-hydroxydecanoate, 3-hydroxytetradecanoate, and 3-hydroxyoctadecanoate. Isolates LS39 and LS46 produced 3-hydroxyoctanoate, 3-hydroxydecanoate, methylated 3-hydroxydodecanoate, and 3-hydroxytetradecanoate.

TABLE 1
Monomers of 3-hydroxyalkanoates produced by
different microbial isolates

| | 3-hydroxyalkanoate monomer | | | | | | | |
|---|---|---|---|---|---|---|---|---|
| Isolate # | C5 | C6 | C8 | C10 | C12 | C14 | C16 | C18 |
| LS1 | − | − | + | + | Methyl | Methyl | − | + |
| LS5 | − | + | + | + | Methyl | Methyl | − | − |
| LS33 | − | − | + | + | − | − | − | − |
| LS34 | − | − | + | + | − | + | − | − |
| LS39 | − | − | + | + | Methyl | Methyl | − | + |
| LS46 | − | − | + | + | Methyl | Methyl | − | − |

### Example 3

### Cell Growth and Production of PHAs by Microbial Isolate LS46

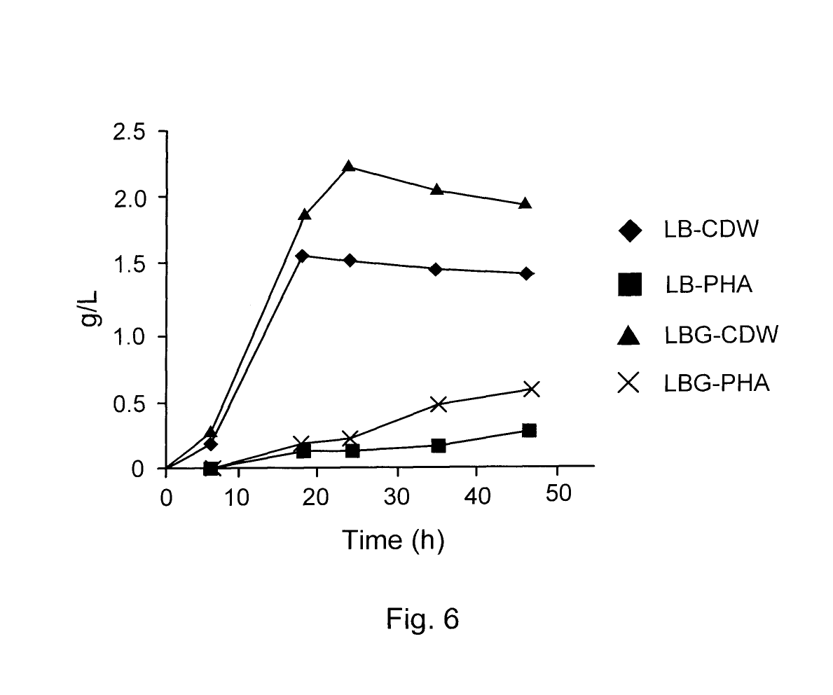

The PHA-producing isolate LS46 was grown in LB liquid medium and in LB liquid medium supplemented with 2% glucose as the carbon source. Flasks containing liquid medium were each inoculated with a 12-h culture of LS 46. Changes in the dry weight of cells and the PHAs produced were monitored for a 48-h period. Total cell dry weights produced by strain LS46 increased in both media types during the first 24 h of incubation (FIG. 6). The presence of PHAs in both types of culture media was detected after 18 h of incubation and steadily increased for the duration of the 48-h culture period (FIG. 6). The rate of PHAs production during the 24-48 h period was greater in the LB medium supplemented with glucose when compared to the unsupplemented LB medium.

### Example 4

### Characterization of PHAs Produced by Different Microbial Isolates in Glucose-Supplemented Liquid Media

The different types of 3-hydroxyalkanoate monomers produced by five microbial isolates (i.e., LS1, LS5, LS33, LS39, LS46) were assessed in Ramsay's minimal salts medium supplemented with 2% glucose as a carbon source. Glucose was sterilized separately and was added to sterilized Ramsey's mineral salts medium. The pH was adjusted to 7.0 and the glucose-supplemented medium was autoclaved for 20 min at 121° C. Flasks containing the glucose-supplemented Ramsey's medium were inoculated with 12-h inoculum cultures that had been grown in LB broth. After a 72-h culture period, the cells produced in each flask were harvested by centrifugation and were washed twice with 0.85% NaCl. The washed cells were dried at 80° C. for 48 h after which their dry cell weights were determined. PHAs were extracted from the dry cells using 100 mL hot chloroform in a Soxhlet apparatus for 6 h. The resulting samples were each concentrated to 2-mL volumes after which, PHAs were precipitated by the addition of 20 mL of cold methanol to each sample. Precipitated PHAs were collected by centrifugation and then dried at 60° C. for 24 h. The compositions of the PHAs were determined by GC-MS after methanolysis following the methods described in Example 2.

Among the five microbial isolates grown in 2% glucose, isolate LS46 produced the highest cell dry weight (6.42 g/L) and isolate LS33 produced the least cell dry weight (3.34 g/L). PHA production was maximum in isolate LS46 (22.61% of cell dry weight) while PHA production was mimum in isolate LS1 (7.78% of cell dry weight). GC-MS analysis of purified PHAs from 2% glucose grown cultures identified 3-hydroxydecanoate as a major component of the PHAs, which accumulated up to 66.6-82.0 mol % in different isolates (Table 2).

TABLE 2
Dry matter production, PHAs production and monomer
composition by microbial strains grown in Ramsey's
minimal salts medium supplemented with glucose.

| | | | 3-hydroxyalkanoate monomer | | | |
|---|---|---|---|---|---|---|
| | | PHA content | content (mol %) | | | |
| Isolate # | CDW* (g/L) | (% CDW) | C6 | C8 | C10 | C12 |
| LS1 | 2.22 + 0.043 | 7.78 | ND | 19.7 | 74.3 | 6.1 |
| LS5 | 2.59 + 0.061 | 16.06 | 1.7 | 26.9 | 66.6 | 4.9 |
| LS33 | 1.93 + 0.035 | 9.66 | 0.9 | 19.8 | 78.5 | 0.7 |
| LS39 | 2.07 + 0.015 | 9.79 | 0.7 | 15.9 | 82.0 | 1.4 |
| LS46 | 2.25 + 0.044 | 22.61 | 0.8 | 10.0 | 79.0 | 3.1 |

*CDW: cell dry weight

### Example 5

### PHAs Production in Minimal Media Supplemented with Oils

Minimal liquid medium containing 1% glucose was supplemented with: (i) canola oil at 0.2, 0.5, 0.7 and 1.0% v/v, (ii) corn oil at 0.2, 0.5, 0.7 and 1.0% v/v, or (iii) olive oil at 0.2, 0.5, 0.7 and 1.0% v/v. Microbial isolate strain LS46 was cultured in LB liquid medium for 12 h and then used as the inoculum for flasks containing oil-amended minimal liquid media. The flasks were cultured for 72 hours after which, the PHAs were recovered from the media and analyzed as described in Example 2.

PHAs production by microbial isolate LS46 in minimal medium with different oil supplementation was greater than the PHA production in minimal medium that was supplemented only 1% glucose. The PHA composition in isolate LS46 gown in 1% glucose was 2% 3-hydroxyhexanoate, 15.7% 3-hydroxyoctanoate, 70.5% 3-hydroxydecanoate and 11.8% 3-hydroxydodecanoate. Addition of different concentrations of oils not only increased PHA production over the minimal media but it also affected the composition of PHA. The optimum concentration of canola and corn oil was 0.7%, which yielded 54.1% and 54.37% PHA, respectively. Olive oil at 1% concentration produced 53.7% PHA. Oil concentrations higher or lower than these concentrations resulted in lowered PHA production. Use of different oils in the culture media affected the types of monomers produced by microbial strain:LS46. Lower concentrations of canola oil (0.2% and 0.5%) resulted in accumulated of relatively more 3-hydroxyoctanoate and 3-hydroxydecanoate, while higher concentrations of canola oil (0.7% and 1.0%) produced more 3-hydroxydodecanoate (Table 3).

TABLE 3
Production of PHA monomers in oil-amended
minimal liquid media

| | | PHA | 3-hydroxyalkanoate | | | |
|---|---|---|---|---|---|---|
| Media | CDW* | content | monomer content (mol %) | | | |
| treatment | (g/L) | (% CDW) | C6 | C8 | C10 | C12 |
| Control (1% | 2.73 + 0.015 | 7.60 | 2.0 | 15.7 | 70.5 | 11.8 |
| glucose) | | | | | | |
| 0.2% canola oil | 2.81 + 0.025 | 37.01 | 2.0 | 14.2 | 57.9 | 25.9 |
| 0.5% canola oil | 3.40 + 0.017 | 50.88 | 1.6 | 12.0 | 51.7 | 34.7 |
| 0.7% canola oil | 2.68 + 0.026 | 54.10 | 1.4 | 9.7 | 48.5 | 40.4 |
| 1.0% canola oil | 2.05 + 0.012 | 45.37 | 1.9 | 9.6 | 45.2 | 43.3 |
| 0.2% corn oil | 2.60 + 0.046 | 29.62 | 2.6 | 12.5 | 49.7 | 35.2 |
| 0.5% corn oil | 2.84 + 0.021 | 36.27 | 2.7 | 11.0 | 44.7 | 41.7 |
| 0.7% corn oil | 2.74 + 0.038 | 54.37 | 1.0 | 9.8 | 34.9 | 55.3 |
| 1.0% corn oil | 1.68 + 0.039 | 41.07 | 1.7 | 9.2 | 37.1 | 52.0 |
| 0.2% olive oil | 3.56 + 0.021 | 25.34 | 2.8 | 10.8 | 45.5 | 40.9 |
| 0.5% olive oil | 3.26 + 0.006 | 43.82 | 2.2 | 7.8 | 34.8 | 55.2 |
| 0.7% olive oil | 2.07 + 0.015 | 51.53 | 2.1 | 8.6 | 36.5 | 52.8 |
| 1.0% olive oil | 1.93 + 0.035 | 53.37 | 2.0 | 9.1 | 38.4 | 50.5 |

Addition of corn oil to the minimal liquid media at different concentrations had similar effects on the monomer composition of PHAs produced by strain LS 34. However, none of the oils at any concentration tested affected production 3-hydroxyhexanoate (C6). Addition of olive oil at 1.0% concentration resulted in maximum production of PHA (53.4%). The compositions of PHAs produced with olive oil were similar to the PHAs produced with corn oil, but were different compared to the compositions of PHA produced with canola oil. These results indicate that PHAs with desired monomer composition can be synthesized by microbial strain LS46 by controlling the ratio of different components e.g., carbon and oils, the growth media.

### Example 6

### Identification of the PHAs-Producing Microbial Isolates

16S rDNA sequencing identified the six PHAs producing microbial isolates disclosed in Example 2. Genomic DNA was isolated from PHA producing bacteria using Wizard® DNA purification kit (Wizard is a registered trademark of Promega Corp., Madison, Wis.). The genomic DNA was amplified using 16S rDNA primers (BAC27 for 5′-GAG TTT GAT CMT GGC TCA G-3′ and BAC1398 rev 5′-CGG TGT GTA CAA GGC CCG GGA ACG-3′ using initial denaturation of 3 min at 94° C., 30 cycles of 45 seconds (s) at 94° C.-30 s at 50° C.-60 s at 72° C. final elongation of 5 min at 72° C. The amplified DNA was extracted from the gel using Gel Extraction kit (Qiagen) and was sequenced using same primers. The sequences were compared with the NCBI database to identify the bacteria using the BLASTn sequence analysis tool. Five of the 6 microbial isolates were identified as Pseudomonas sp. while the sixth microbial isolate was identified as a Comamomas sp. The 16S rDNA of microbial strains LS1, LS5 and LS33 showed 99% sequence identity with Pseudomonas putida strain WXY-19 (Table 4). The 16S rDNA of microbial isolate LS39 showed 99% sequence identify to Pseudomonas plecoglossicida strain 3, while the 16S rDNA of microbial isolate LS46 had 99% sequence identity with Pseudomonas sp. 1008 (Table 4). Microbial isolate LS34 had a 98% sequence identity with Comamonas spp NH-3.

TABLE 4
Identification of mcl-PHAs producing bacteria by 16S rDNA
gene sequencing

| Isolate | | | | % |
|---|---|---|---|---|
| # | 1^{st} match* | 2^{nd} match* | 3^{rd} match* | homology |
| LS1 | P. putida | P. putida P-19 | Pseudomonas | 99 |
| | WXY-19 | | sp. SP-2 | |
| LS5 | P. putida WXY | P. putida P | Pseudomonas | 99 |
| | | | sp. SP-2 | |
| LS33 | P. putida WXY | P. putida P | Pseudomonas | 99 |
| | | | sp. SP-2 | |
| LS34 | Comamonas sp. | C. tetosteroni | C. tetosteroni | 98 |
| | NH-3 | | H-18 | |
| LS39 | P. | P. | Pseudomonas | 99 |
| | plecoglossicida | plecoglossicida | 142510 | |
| | st-3 | st-3 | | |
| LS46 | Pseudomonas sp. | Pseudomonas | Pseudomonas | 99 |
| | KB08 | sp. KA08 | sp. JA4 | |

*1^{st}, 2^{nd}, and 3^{rd} matches are based on the top three highest E values determined by BLASTn analysis

### Example 7

### Effects of Nitrogen Nutrition on Production of PHAs by P. putida LS46

Effects of nitrogen nutrition on production of PHAs by P. putida LS46 were assessed under culture conditions with excess nitrogen availability and under culture conditions with limited nitrogen availability. Excess nitrogen availability conditions were provided by adding 4 g/L of NH₄SO₄ to minimal liquid medium supplemented with 2% glucose. Adding 1 g/L of NH₄SO₄ to minimal liquid medium supplemented with 2% glucose provided limited nitrogen availability conditions. Flasks containing the liquid medium were inoculated with microbial isolate LS46 as disclosed in the previous examples and then incubated for 72 h.

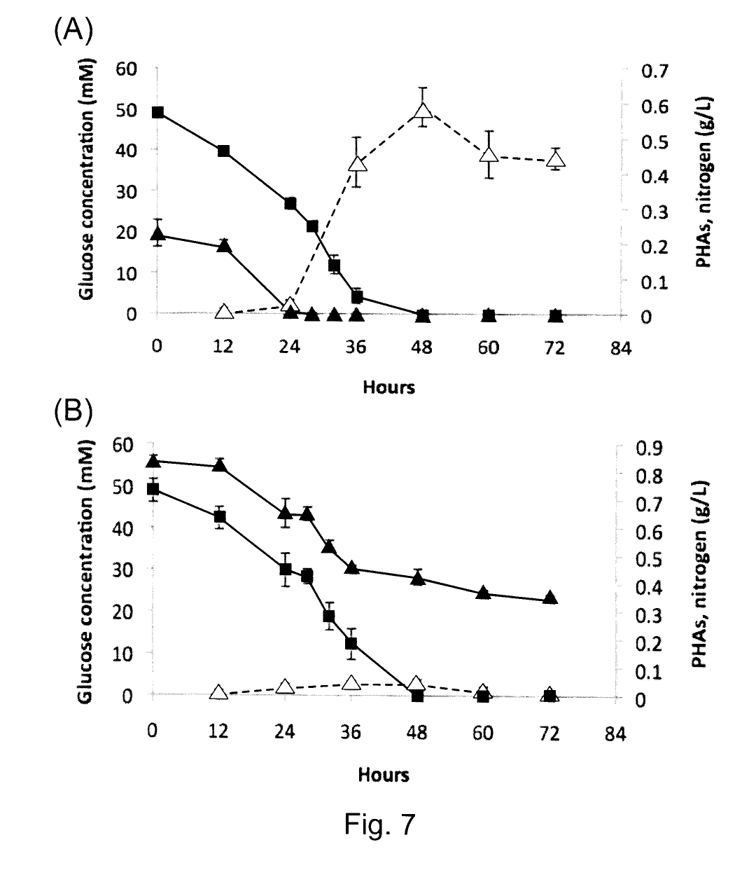

The data in FIG. 7(A) show that under nitrogen limiting conditions, the glucose concentration decreased from 50 mM to 4.42 mM after 36 h (filled squares) while NH₄⁺ decreased from 0.23 g/L to 0.01 g/L during the first 24 h of incubation, and then was undetectable for the duration of the 72-h culture period. The data in FIG. 7(B) show that under nitrogen excess conditions, the glucose concentration decreased from 50 mM to 12.25 mM after 36 h (filled squares) while NH₄⁺ decreased from 0.84 g/L to 0.65 g/L during the first 24 h of incubation. At the end of the 72-h culture period, three was still 0.35 g/L of NH₄⁺ present in the excess nitrogen liquid medium indicating that nitrogen levels were not limiting during this time course (filled triangles). There were no differences in the cell dry weight (cdw) of P. putida LS46 cells produced in nitrogen-excess conditions and nitrogen limited-conditions at the end of the 72-h culture period. mcl-PHAs production was detected after 12 h in both nitrogen-limited and nitrogen-excess conditions (open triangles in FIGS. 7(A) and 7(B) respectively). There was a 6-fold increase in mcl-PHA production after 48 h in the nitrogen-limiting medium (FIG. 7(A)). However, under nitrogen excess condition, the increase in pHAs production was only 1.95 fold after 48 h (FIG. 7(B)).

### Example 8

### Effects of Saturated Fatty Acid Supplementation on Production of PHAs by P. putida LS46

Ramsey's liquid medium was supplemented with one of 20 nM octanoic acid, decanoic acid, dodecanoic acid, and tetradecanoic acid. Flasks containing the supplemented liquid media were then each inoculated with 1% inoculum of P. putida LS46 and then cultured for 48 h. Cultures were then harvested and their cdw, the percentage accumulation of mcl-PHAs in the cells, and the mol % monomer composition of C8 (20 mM) the mcl-PHAs were determined. In comparison to PHAs production on glucose, P. putida LS46 grown on fatty acids produced more mcl-PHAs (Table 5). The mcl-PHAs production was 56.1, 36.9, 31.6, and 26% of the cdw when cultured in media containing 20 mM octanoic, decanoic, dodecanoic, and tetradecanoic acid respectively (Table 5). The monomer composition of the mcl-PHAs produced on different fatty acids was different than that of the mcl-PHAs produced on glucose. In glucose medium, 3-hydroxydecanoate was the major component (68.8%), while the mcl-PHAs produced on octanoic acid contained 88.5% 3-hydroxyoctanoate and only 3.9% 3-hydroxydecanoate. The PHAs produced on decanoic acid consisted of 50.4% 3-hydroxyoctanoate and 48% 3-hydroxydecanoate. The mcl-PHAs produced on dodecanoic acid consisted of 25.8% 3-hydroxyoctanoate, 47.3% 3-hydroxydecanoate, and 24.0% dodecanoic acid (Table 5).

TABLE 5
Identification of mcl-PHAs producing bacteria by
16S rDNA gene sequencing

| | | | 3-hydroxyalkanoate | | | |
|---|---|---|---|---|---|---|
| | CDW | PHA content | monomer content (mol %) | | | |
| C source | (g/L) | (% CDW) | C6 | C8 | C10 | C12 |
| Glu (2%) | 2.68 | 22.6 | 1.7 | 14.7 | 68.8 | 14.9 |
| C8 (20 mM) | 2.49 | 56.1 | 6.5 | 88.5 | 3.9 | 1.1 |
| C10 (20 mM) | 3.51 | 36.9 | 1.5 | 50.4 | 48.0 | 1.1 |
| C12 (20 mM) | 3.12 | 31.6 | 2.8 | 25.8 | 47.3 | 24.0 |
| C14 (20 mM) | 3.11 | 26.0 | ND* | 39.6 | 35.6 | 24.8 |

*ND: not detected

### Example 9

### Effects of Salicylic Acid on Production of PHAs by P. putida LS46

Salicylic acid has been used as an inhibitor of β-oxidation in plant, animal, and bacterial cells. It has been observed that addition of salicylic acid between 5 mM and 60 mM in growth medium altered the monomer composition of mcl-PHAs produced by P. aeruginosa. Salicylic acid at 10 mM inhibited P. putida LS46 growth. Therefore, 1-5 mM concentration of salicylic acid was studied for its effect on mcl-PHA production by P. putida LS46. To separate volumes of Ramsey's liquid medium supplemented with 2% of glucose were added: (i) no salicylic acid (control), (ii) 1.0 nM salicylic acid, (iii) 2.5 mM salicylic acid, or (iv) 5.0 salicylic acid. After sterilization, the liquid media were dispensed into flasks which were then each inoculated with 1% inoculum of P. putida LS46, and then cultured for 48 h. Cultures were then harvested and their cdw, the percentage accumulation of mcl-PHAs in the cells, and the mol % monomer composition of C8 (20 mM) the mcl-PHAs were determined. The data in Table 6 show that addition of 2.5 mM salicylic acid not only improved mcl-PHA production, it also changed the monomer composition. 3-hydroxyoctanoate decreased, while 3-hydroxydecanoate increased, in the presence of 2.5 mM and 5.0 mM salicylic acid, compared with the control (Table 6).

TABLE 6
Effects of salicylic acid on production of mcl-PHAs
by P. putida LS46

| | | | 3-hydroxyalkanoate | | | |
|---|---|---|---|---|---|---|
| Salicylic acid | CDW | PHA content | monomer content (mol %) | | | |
| (mM) | (g/L) | (% CDW) | C6 | C8 | C10 | C12 |
| 0 | 3.21 | 25.53 | 5.15 | 45.24 | 47.90 | 1.91 |
| 1.0 | 3.08 | 26.07 | 3.97 | 46.30 | 47.90 | 1.91 |
| 2.5 | 2.89 | 28.90 | 3.39 | 44.06 | 52.03 | 0.93 |
| 5.0 | 2.64 | 21.89 | 2.46 | 42.49 | 53.36 | 0.66 |

### Example 10

### Comparison of the P. putida LS46 Genome with Other P. putida Genomes

The complete genome of P. putida LS46 was sequenced and annotated using Joint Genome Institute software. The P. putida LS 46 genome was compared with other five strains of P. putida: (i) P. putida KT2440, (ii) P. putida F1, (iii) P. putida BIRD-1, (iv) P. putida GB-1, and (v) P. putida W619. The genome statistics indicated that P. putida LS46 had a GC ratio of 62%, which was identical to four P. putida strains i.e., P. putida KT2440, P. putida BIRD-1, P. putida F1 and P. putida GB-1 (Table 7). However, P. putida strain W619 had a lower GC ratio of 61%. The P. putida LS46 genome size was 6,034,795 bp which smaller than genomes of P. putida GB-1 and P. putida KT2440, but larger than P. putida F1 and P. putida W619. The P. putida LS46 genome was found to encode 5304 genes, while the other four strains (F1, GB-1, KT2440, and W619) encoded 5423, 5515, 5481, and 5292 genes, respectively (Table 7). Like wise, the COG counts were different among the different P. putida strains.

TABLE 7
Comparison of P. putida LS 46 genome with other P. putida genomes

| | Pseudomonas putida strain | | | | | |
|---|---|---|---|---|---|---|
| Character | LS46 | F1 | GB-1 | KT2440 | W619 | BIRD-1 |
| Number of bases | 6034795 | 5959964 | 6078430 | 6181860 | 5774330 | 5731541 |
| Number of CDS | 5304 | 5300 | 5417 | 5420 | 5471 | 4960 |
| CDS with predicted | 78.15 | 73.5 | 74.9 | 76.9 | 70 | 75.23 |
| function (%) | | | | | | |
| CDS without function with | 20.7 | 23.6 | 22.7 | 19.1 | 25.6 | 23.7 |
| homolog (%) | | | | | | |
| CDS without function | ND | 0.65 | 0.7 | 4.0 | 4.4 | 0.55 |
| without homlog (%) | | | | | | |
| COG | 4105 | 4171 | 4267 | 4199 | 4089 | 4089 |
| COG (%) | 77.39 | 76.91 | 77.37 | 76.61 | 77.27 | 82.03 |
| Pseudogenes | 0 | 49 | 8 | ND | 12 | 0 |
| Putative ortholog relation | 100 | 86.62 | 83.83 | 87.91 | 79.5 | 79.0 |
| (%) | | | | | | |
| % GC | 61.71 | 61 | 62 | 62 | 61 | 61.74 |
| tRNA | 60 | 76 | 74 | 73 | 75 | 64 |

### Example 11

### Comparison of P. putida LS46 Genome on the Basis of House Keeping Genes

Twenty-eight (28) house-keeping genes of P. putida LS46 were compared with other P. putida genomes used by Zeigler (2003, Int. J. Syst. Evol microbial. 53: 1893-1900) to study genome sequence identity (Table 8). The house-keeping genes showed 94-100% nucleotide sequence identity with P. putida F1, KT2440, BIRD-1, GB-1, and W619 genes. P. putida LS46 was closely related P. putida F1, and showed 100% homology with dxs, cpn60, glyA, argS, ffh, ftsZ, metK, uvrB, atpD, dnaB, dnaJ, rho, recA, rpoA, pgk, and uvrC (Table 8).

TABLE 8
Comparison of P. putida LS46 genome on the basis of house keeping genes

| | | Homology (%) | | | | |
|---|---|---|---|---|---|---|
| Gene | Product | F1 | BIRD-1 | KT2440 | W619 | GB-1 |
| dxs | Deoxyxylulose-phosphate | 100 | 99.84 | 99.68 | 98.57 | 99.21 |
| | synthase | | | | | |
| recN | Recombination repair protein | 99.82 | 99.28 | 99.28 | 94.79 | 97.49 |
| cpn60 | Heat shock protein 60 | 100 | 100 | 99.82 | 98.72 | 95.35 |
| thdF/trmE | GTP binding, Thiophene | 99.78 | 99.76 | 99.78 | 96.93 | 98.25 |
| | oxidation | | | | | |
| trpS | Tryptophan tRNA synthetase | 98.20 | 97.31 | 97.76 | 96.19 | 93.50 |
| glyA | Serine hydroxymethyltransferase | 100 | 100 | 100 | 98.56 | 100 |
| lepA | GTP binding membrane protein | 99.83 | 99.83 | 99.83 | 98.83 | 99.16 |
| argS | Ariginine tRNA synthetase | 100 | 99.65 | 99.48 | 96.99 | 96.81 |
| ffh | GTP binding export factor | 100 | 100 | 100 | 99.78 | 99.34 |
| serS | Serine tRNA synthetase | 99.77 | 99.53 | 99.77 | 97.65 | 98.36 |
| ftsZ | Tubulin like division protein | 100 | 100 | 100 | 100 | 99.75 |
| metK | Methionine adenosyltransferase | 100 | 100 | 100 | 93.23 | 98.74 |
| atpA | ATP synthase F1 α unit | 99.61 | 100 | 99.61 | 98.64 | 99.81 |
| dnaX | DNA polymerase III subunit Y, τ | 99.13 | 95.52 | 96.1 | 84.55 | 92.49 |
| uvrB | Excision nuclease subunit B | 100 | 99.85 | 99.85 | 97.62 | 99.11 |
| atpD | ATP synthase F1, β subunit | 100 | 100 | 100 | 96.94 | 99.56 |
| aspS | Aspartate tRNA synthetase | 99.83 | 99.83 | 99.83 | 99.15 | 99.66 |
| cysS | Cystteine tRNA synthetase | 98.99 | 99.75 | 99.24 | 97.47. | 98.23 |
| uvrC | Exonuclease ABC, C subunit | 100 | 99.67 | 99.51 | 96.21 | 98.80 |
| ruvB | Holliday juction helicase subunit A | 99.43 | 99.71 | 99.71 | 96.84 | 98.28 |
| metG | Methionine tRNA synthetase | 99.71 | 99.41 | 99.85 | 97.5 | 98.67 |
| dnaB | Replicative DNA helicase | 100 | 99.78 | 99.78 | 98.28 | 98.49 |
| dnaJ | Chaperone with DnaK | 100 | 98.67 | 98.67 | 98.66 | 99.73 |
| rho | Transcription termination factor | 100 | 100 | 100 | 100 | 100 |
| proS | Proline tRNA synthetase | 99.82 | 99.30 | 99.47 | 97.55 | 97.55 |
| recA | DNA strand exchangeand | 100 | 100 | 100 | 99.09 | 98.31 |
| | renaturation | | | | | |
| rpoA | RNA polymerase | 100 | 100 | 100 | 100 | 100 |
| eno | Enolase | 100 | 100 | 99.53 | 99.53 | 99.53 |
| lig | DNA ligase | 99.7 | 98.2 | 99.1 | 88.52 | 93.69 |
| pgi | Glucose phosphate isomerase | 99.82 | 99.28 | 99.64 | 98.01 | 98.17 |
| tig | Trigger factor | 100 | 99.54 | 99.77 | 95.19 | 97.9 |
| pgk | Phosphoglycerate kinase | 100 | 98.97 | 99.74 | 97.67 | 99.74 |

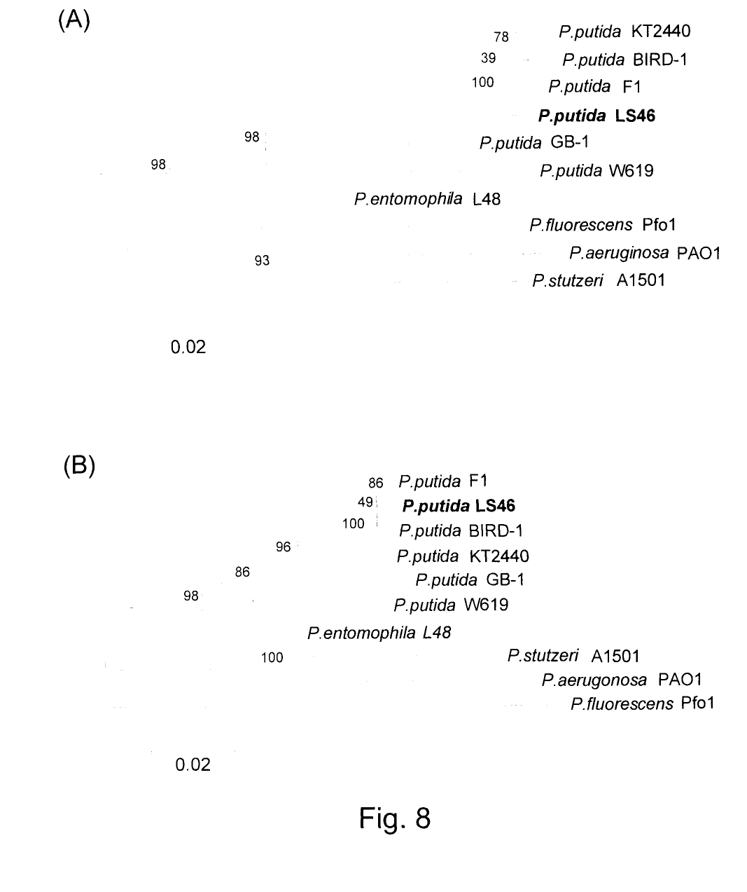

Similar house-keeping genes were also present in P. putida GB-1 (99.3-99.6% homology) and P. putida W619 (94.9-99.6% homology). The neighbor-joining tree of cpn60 and recA indicated that P. putida LS46 was very closely related to P. putida F1. However, it was possible to differentiate P. putida LS46 from the other P. putida strains on the basis of recA (FIG. 8(A)) and cpn60 gene sequences (FIG. 8(B)).

### Example 12

### Identification of Genes in P. putida LS46 Associated with Mcl-PHA Production from Glucose or Fatty Acids

Fatty acid synthesis or β-oxidation of fatty acids provides the substrates for accumulation of PHA. Therefore, the genes for de novo fatty acid synthesis or β-oxidation are closely associated with PHA production. The genetic manipulations of these genes have been used to increase mcl-PHA production or to produce mcl-PHA with desired monomer composition. Some of these genes have more than one copy. The genes associated with mcl-PHA production are listed in Table 9. The fatty acid synthesis enzymes (Fab proteins) and fatty acid degradation enzymes (Fad proteins) of P. putida LS46 had more than 90% homology with Fab/Fad proteins from other 4 strains of P. putida. Even gene products from phaC1, phaZ, phaC2, phaD, phaF and phal, which are directly, involved in mcl-PHA production showed 92-100% homology with protein from other P. putida strains.

TABLE 9
Identification of P. putida LS46 gene products associated with PHA
production and their homology to protein from other P. putida strains

| | Locus | | Size | KT2 | | | |
|---|---|---|---|---|---|---|---|
| Enzyme | tag | Protein | (aa) | 440 | GB-1 | F ! | W619 |
| ¹Poly(R)-hydroxyalkanoic acid | 34530 | PhaC1 | 559 | 99 | 98 | 99 | 94 |
| synthase | | | | | | | |
| Poly(3-hydroxyalkanoate) | 34520 | PhaZ | 283 | 99 | 99 | 100 | 95 |
| depolymerase | | | | | | | |
| Poly(R)-hydroxyalkanoic acid | 34510 | PhaC2 | 560 | 99 | 96 | 99 | 92 |
| synthase | | | | | | | |
| Transcriptional regulator | 34500 | PhaD | 204 | 99 | 96 | 99 | 95 |
| PHA granule associated protein | 34490 | PhaF | 253 | 99 | 97 | 100 | 94 |
| PHA granule associated protein | 34480 | PhaI | 139 | 96 | 92 | 96 | 90 |
| ²Enoyl-CoA hydratase/3- | 44210 | PhaJ | 229 | 99 | 99 | 99 | 99 |
| hydroxyacyl-CoA dehydrogenase | | | | | | | |
| Acyl dehydratase | 11850 | PhaJ1 | 156 | 100 | 99 | 100 | 96 |
| Hydroxyacyl-ACP:CoA transacylase | 40380 | PhaG | 295 | 99 | 98 | 99 | 89 |
| Acyl-CoA synthetase | 11880 | FadD | 565 | 99 | 99 | 100 | 97 |
| 3-ketoacyl-CoA thiolase | 46790 | FadA | 392 | 99 | 99 | 100 | 98 |
| Acetyl-CoA acetyltransferases | 47590 | FadAx | 380 | 94 | 92 | 94 | 91 |
| 3-hydroxyacyl-CoA dehydrogenase | 46780 | FadB | 715 | 99 | 99 | 99 | 99 |
| Acyl-CoA synthetase | 11870 | FadD2 | 562 | 99 | 98 | 99 | 93 |
| Acyl CoA dehydrogenase | 02200 | FadE | 601 | 99 | 99 | 99 | 97 |
| ³Malonyl CoA-acyl carrier protein | 44980 | FabD | 312 | 98 | 97 | 100 | 92 |
| transacylase | | | | | | | |
| 3 hydroxyacyl(decanyl)-ACP | 15300 | FabA | 171 | 100 | 99 | 98 | 98 |
| dehydratase | | | | | | | |
| 2-Oxoacyl carrier protein synthase | 15290 | FabB | 406 | 99 | 99 | 99 | 99 |
| 3-ketoacyl-(acyl-carrier-protein) | 04470 | FabG | 450 | 100 | 99 | 99 | 99 |
| reductase | | | | | | | |
| 3-oxoacyl-(acyl-carrier-protein) | 44490 | FabG | 450 | 93 | 91 | 92 | 92 |
| reductase | | | | | | | |
| 3-hydroxyacyl-[acyl carrier protein] | 41750 | FabZ | 146 | 100 | 100 | 100 | 95 |
| dehydratase | | | | | | | |

¹PHA synthesis genes;
²Fatty acid synthesis enzymes;
³Fatty acid degradation enzymes

### Example 13

### Organization of pha Synthesis Genes in P. putida LS46

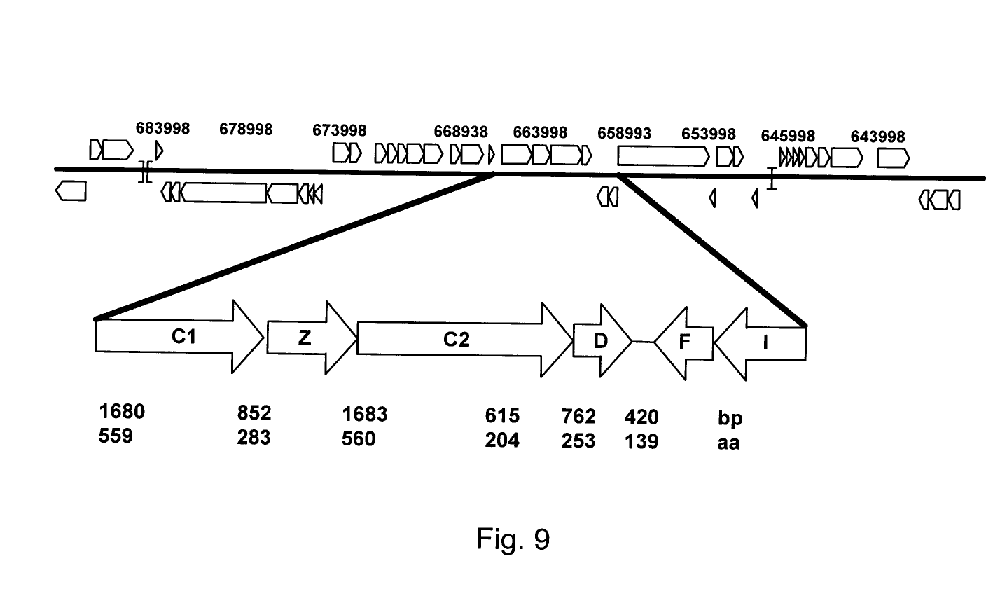

The complete genome sequence of P. putida LS46 indicated that pha operon contained six genes: phaC1, phaZ, phaC2, phaD, phaF, and phal (FIG. 9). Of these, phaC1, phaZ, phaC2, and phaD were transcribed in one direction, while phaF and phal were transcribed in opposite direction (FIG. 9). The organization of pha genes in P. putida LS46 was identical to other P. putida strains (KT2440, F1, GB-1, and W619). The phaC1 and phaC2 genes encode PHA synthase while phaZ encodes PHA depolymerase. The phaD, phaF and phal are regulatory genes.

### Example 14

### Construction of Plasmids with Deletion of phaC1, phaZ, and phaC2 Genes in Plasmid pPK49

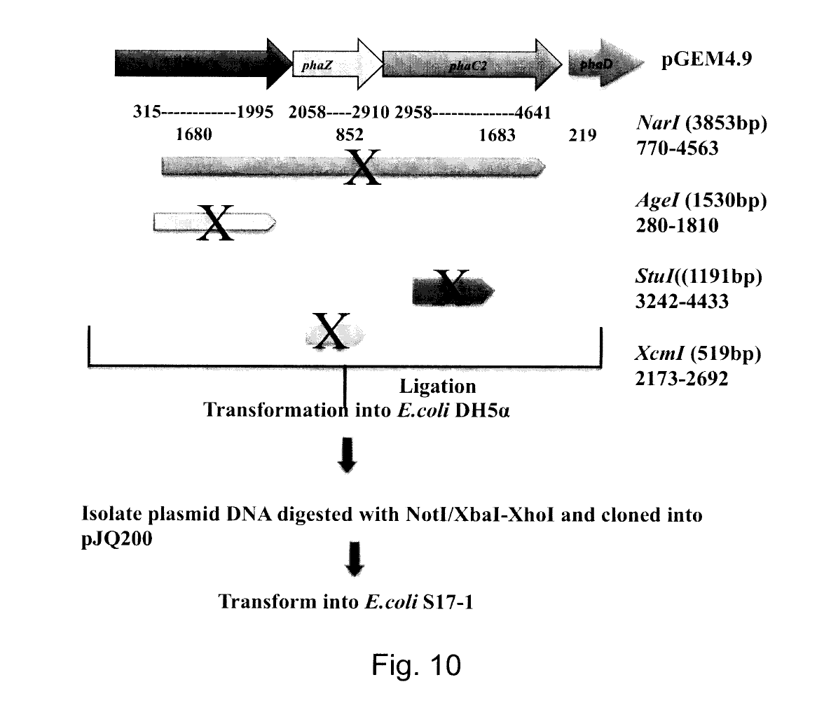

Plasmid pPK49 was digested with NarI, gel purified, religated and transformed into E. coli DH5. NarI digestion deleted 3.8 kb region from plasmid pPK49 containing phaC1, phaZ and phaC2 genes. The plasmid with NarI deletion was designated as pPKN49. A 770 bp region on left flanking region and 250 bp on right flanking region was present on the pPKN49. The plasmid pPKN49 was digested with NotI and a 1.1 kb fragment was gel purified and cloned into NotI linearized, CIP treated plasmid pJQ200. The resulting plasmid pPKΔ38 was transformed into E. coli S17-1 (FIG. 10).

For deletion of phaC1 gene, plasmid pPK49 was digested with AgeI, gel purified, religated and transformed into E. coli DH5a. This step deleted a 1521 bp (340 to 1861) fragment from pPK49 carrying phaC1 gene (FIG. 10). This plasmid was designated pPKA49. A 1.6 kb NotI fragment from pPKA49 was cloned into NotI linearized, CIP treated pJQ200. This plasmid was designated as pPKΔC1 and was transformed to E. coli S17-1. For deletion of phaC2, a 2.8 kb NheI-XhoI fragment of pPK49 carrying phaC2 gene was cloned into Nde1-XhoI digested pET28a plasmid. The resulting plasmid pET28.28 was digested with StuI, which deleted 1191 bp of phaC2 gene (3242-4433). The plasmid with deletion was religated and transformed into E. coli DH5a. A 1.6 kb XhoI-XbaI fragment from this plasmid was cloned into XhoI-XbaI digested pJQ200. This plasmid was designated as pPKΔC2 and transformed into E. coli S17-1.

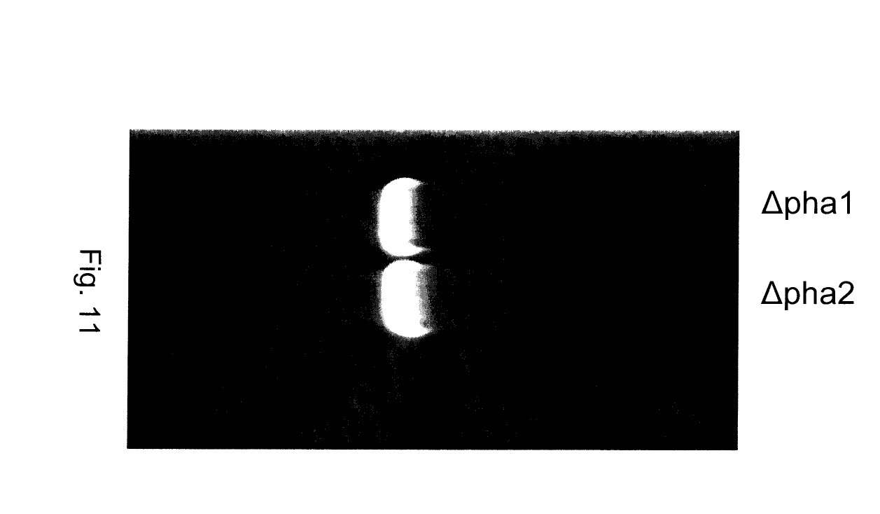

To delete phaZ, pPK49 was digested with Xcm/, religated and transformed into E. coli DH5a. There was deletion of 519 bp in phaZ gene. A 0.9 kb NotI-EcoRI fragment was cloned into pET28. From this plasmid a 1.0 kb XhoI-XbaI fragment was cloned into pJQ200 to give pJQ200ΔZ. Confirmation of the 3.8 kb deletion in the pha region of plasmid pJQ200ΔZ was confirmed by PCR amplification (FIG. 11).

For confirmation of deletions in phaZ, gene primers phaZfor 5′-GCCGCAACCCTATATTTTC A-3′ and phaZrev 5′-GGGTGATCAGGAACAGATGG-3′ were used to amplify the phaZ gene. A 300 bp fragment was amplified from the plasmid with deletion in phaZ gene (pJQ200ΔZ) as compared to an 800 by fragment that was amplified from plasmid papGem49, confirming a deletion of 500 bp. For confirmation of phaC2 deletion, phaC2 for 5′-GAGACATATGATGAAAGACAAACCGGCCAAAG-3′ and phaC2rev 5′-TATATCTAGAAGGGCGCATTCAAG GATAC-3′ were used. These primers amplified a 1.7 kb fragment in pGEM49, but amplified only 500 bp fragment in plasmid pJQ200AC2, confirming a deletion of 1.2 kb in the phaC2 gene.

### Example 16

### Construction P. putida LS46 Mutants with phaZ Deletion

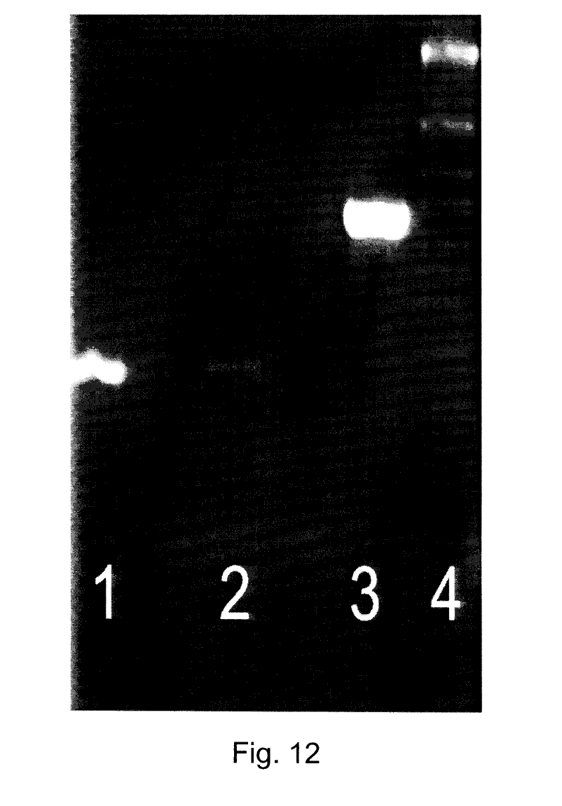

Plasmid pJQ200ΔZ was transformed into E. coli S17-1 and conjugated with P. putida LS46 by biparental mating. Exconjugants were screened on LB plates containing Cm₅₀Gm₁₀₀. Five exconjugants were purified grown in LB broth and serial dilutions were plated on LB containing 10% sucrose. The clones growing on sucrose plates were screened for the loss of gentamycin marker. Screening of 250 clones gave 6 gentamycin sensitive clones. Genomic DNA was isolated from these clones and deletion in phaZ gene was confirmed by amplification of phaZ gene using primers phaZfor 5′-GCCGCAACCCTATATTTTCA-3′ and phaZrev 5′-GGGTGAT CAGGAACAGATGG-3′. A 300 bp fragment of phaZ was amplified in the phaZ deletion mutant Z502, as compared to 800 bp fragment in P. putida LS46 (FIG. 12). A 519 bp deletion in phaZ gene of P. putida LS46 was also generated using XcmI enzyme. This deletion mutant was designated P. putida strain LS46 Z502, and subsequently referred to herein as P. putida strain Z502.

### Example 17

### mcl-PHA Production P. putida LS46 phaC1 and phaZ Deletion Mutants

mcl-PHAs are synthesized from glucose or from fatty acid acids. The contribution of phaC1 and phaZ gene in PHA production was studied by contructing phaC1 and phaZ deletion mutants of P. putida strain LS46. A 1530 bp AgeI fragment of containing phaC1 gene was deleted using a pJQ200 suicidal plasmid containing sacB gene. Mcl-PHAs production in a LS46 mutant with a phaC1 deletion named P. putida strain Pc-1-5, was compared with parent P. putida LS46. P. putida strain Pc-1-5 produced 53.6% PHAs in 2% glucose medium after 96 h as compared to parent strain. The monomer composition of PHAs was also different in the P. putida strain Pc-1-5 deletion mutant. The mcl-PHAs of the P. putida strain Pc-1-5 mutant contained 24.7 mol % C8, 66.3 mol % C10, and 9.4 mol % C12, while the mcl-PHAs of the parent P. putida LS46 consisted of 20.2 mol % C8, 71.3 mol % C10, and 8.4 mol % C12 (Table 10).

TABLE 10
mcl-PHA production in phaC1 deletion mutant C1-5 of P. putida LS46

| | | | 3-hydroxyalkanoate | | |
|---|---|---|---|---|---|
| P. putida | | PHA content | monomer content (mol % | | |
| strain # | CDW (g/L) | (% CDW) | C6 | C8 | C10 |
| LS46 | 2.62 + 0.15 | 15.17 + 1.87 | 20.26 + 2.47 | 71.31 + | 8.43 + |
| | | | | 4.37 | 1.99 |
| Pc-1-5 | 2.13 + 0.07 | 8.14 + 0.59 | 24.27 + 0.95 | 66.33 + | 9.40 + |
| | | | | 0.99 | 1.07 |

### Example 18

### mcl-PHA Production by phaZ Deletion Mutant P. putida Strain Z502

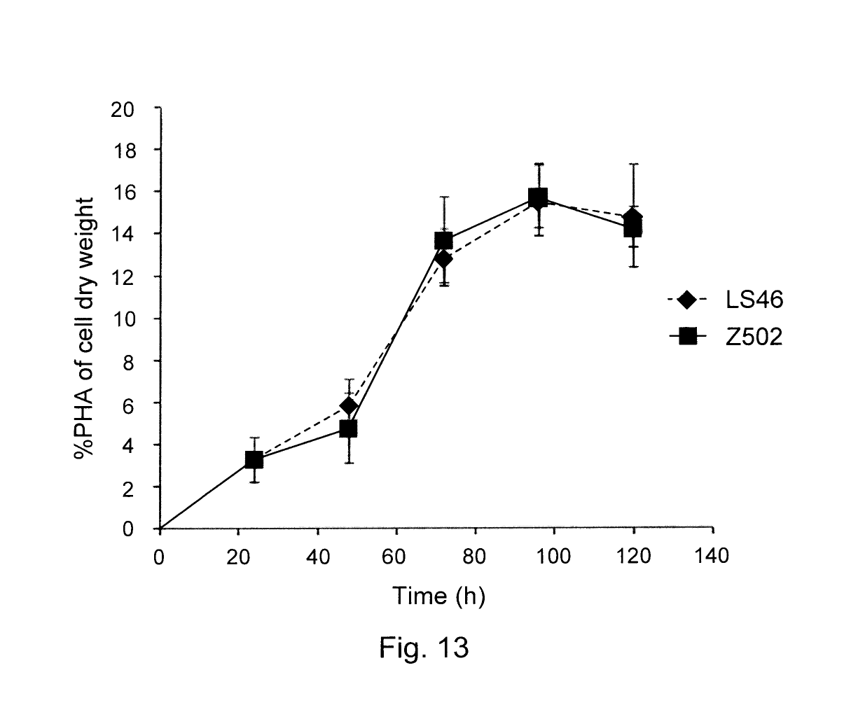

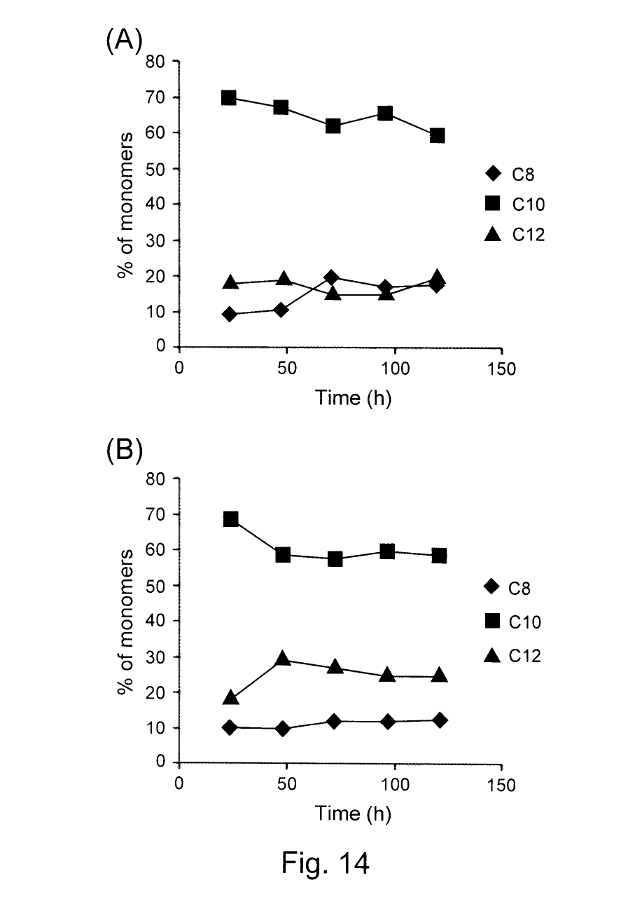

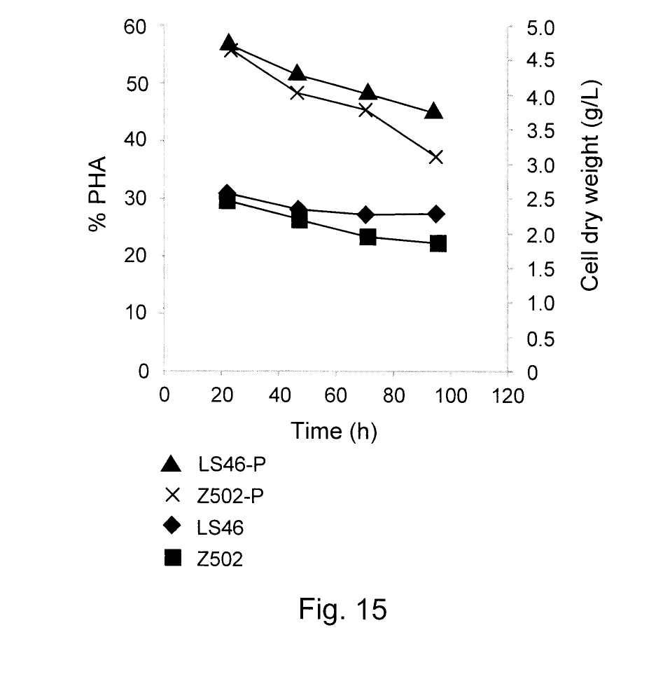

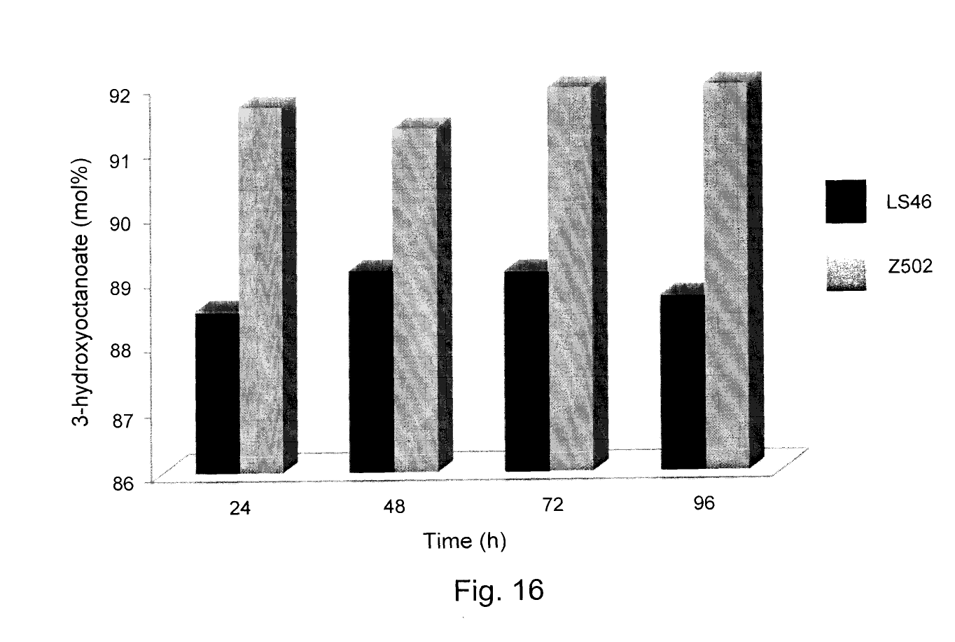

The deletion of phaZ gene in the P. putida strain Z502 mutant did not affect production of mcl-PHAs (FIG. 13) or the or monomer composition (FIG. 14 (B)) compared with P. putida LS46 (FIGS. 13 and 14(A)) when grown in medium supplemented with 2% glucose. mcl-PHA production by mutant P. putida strain Z502 was studied using octanoic acid as the sole carbon source. Ramsay's medium supplemented with 20 mM octanoic acid was inoculated with a 1% culture of P. putida LS46. Samples were drawn after 24, 48, 72 and 96 h after inoculation. Cell dry weight (CDW) and % PHA of CDW were calculated. Monomer composition was determined by GC analysis after methanolysis. Data indicated that cdw in both the parent strain, P. putida LS46, and the phaZ mutant strain, P. putida strain Z502, decreased with incubation (FIG. 15). In the case of P. putida LS46, cdw decreased to 91.2, 87.7 and 88.6% after 48, 72, and 96 h, respectively. Cdw in P. putida strain Z502 decreased to 89.1, 78.7, and 74.7% after 48, 72 and 96 h, respectively. This indicated that mutation in phaZ gene affected the cell survival in octanoic medium and cells with the phaZ mutant dying earlier than parent P. putida LS46. The P. putida strain Z502 mutant produced 54.25%, 47.22%, 44.10% and 35.91% PHAs of CDW after 24 h, 48 h, 72 h and 96 h respectively. In comparison, P. putida LS46 produced 56.2%, 50.1%, 50.4%, and 49.0% PHAs after 24 h, 48 h, 72 h, and 96 h, respectively. The phaZ gene is required to breakdown the storage compound during stationary phase and cell viability is affected if PHAs is not recycled. Due to earlier cell lysis, the mcl-PHA content of the phaZ mutant was less than parent P. putida LS46. The mol % of 3-hydroxyoctanoate monomer in the mcl-PHA biopolmer produced by Z502 in octanoic acid medium was higher than the mol % 3-hydroxyoctanoate monomer in the mcl-PH biopolymer synthesized by P. putida LS46 (FIG. 16).

### Deposit of Microorganisms

Samples of isolates P. putida strains LS1, LS5, and LS 46 as disclosed herein were deposited at the INTERNATIONAL DEPOSITORY AUTHORITY OF CANADA (IDAC) of 1015 Arlington Street, Winnipeg, Manitoba, Canada, R3E,3R2 (Telephone: 204-789-6030; Fax: 204-789-2018) for patent purposes under the terms of the Budapest Treaty. The deposits were made on Feb. 23, 2005, and the deposit receipt numbers are IDAC 181110-01 (P. putida isolate LS1), IDAC 181110-02 (P. putida isolate LS5), and IDAC 181110-03 (P. putida isolate LS46). Samples of P. putida strains Pc-1-5 and Z502 were deposited with IDAC on Nov. 17, 2011 for patent purposes under the terms of the Budapest Treaty. The deposit receipt numbers are IDAC 171111-01 (P. putida isolate Pc-1-5), IDAC 171111-02 (P. putida isolate Z502).

1. A process for producing medium-chain-length 3-hydroxyalkanoic acids in a liquid medium, the process comprising the steps of:
(a) intermixing in a reaction vessel a liquid culture medium and a microbial inoculum comprising a culture of a Pseudomonas putida strain selected from the group consisting of IDAC 181110-01, IDAC 181110-02, IDAC 181110-03, IDAC 171111-01, and IDAC 171111-02;
(b) culturing the mixture for a period of time to allow for microbial cell growth to occur in the liquid culture medium thereby producing: (i) a plurality of microbial cells wherein medium-chain-length 3-hydroxyalkanoic acids are synthesized, and (ii) a spent liquid medium;
(c) separating the medium-chain-length 3-hydroxyalkanoic acids from the plurality of microbial cells and the spent liquid medium; and
(d) recovering the separated medium-chain-length 3-hydroxyalkanoic acids.

2. The process of claim 1, wherein the process further comprises purifying the recovered medium-chain-length 3-hydroxyalkanoic acids.

3. The process of claim 1, wherein the process further comprises separating the recovered medium-chain-length 3-hydroxyalkanoic acids into two or more of a C6 polyhydroxyalkanoate monomer, a C8 polyhydroxyalkanoate monomer, a C10 polyhydroxyalkanoate monomer, a C12 polyhydroxyalkanoate monomer, and a C14 polyhydroxyalkanoate monomer.

4. The process of claim 1, wherein the liquid culture medium is supplemented with a 5-carbon monosaccharide selected from the group consisting of arabinose, lyxose, ribose, xylose, ribulose, xylulose, and combinations thereof.

5. The process of claim 4, wherein the 5-carbon monosaccharide is provided at a concentration from the range of about 0.5% to about 30%.

6. The process of claim 4, wherein the 5-carbon monosaccharide is provided at a concentration from the range of about 1% to about 15%.

7. The process of claim 1, wherein the liquid culture medium is supplemented with a 6-carbon monosaccharide selected from the group consisting of glucose, fructose, mannose, galactose, allose, altose, idose, and talose, and combinations thereof.

8. The process of claim 7, wherein the 5-carbon monosaccharide is provided at a concentration from the range of about 0.5% to about 30%.

9. The process of claim 7, wherein the 5-carbon monosaccharide is provided at a concentration from the range of about 1% to about 15%.

10. The process of claim 1, wherein the liquid culture medium is supplemented with a disaccharide selected from the group consisting of sucrose, lactose, lactulose, maltose, trehalose, and combinations thereof.

11. The process of claim 10, wherein the disaccharide is provided at a concentration from the range of about 0.5% (w/w) to about 30% (w/w).

12. The process of claim 10, wherein the disaccharide is provided at a concentration from the range of about 1% (w/w) to about 15% (w/w).

13. The process of claim 1, wherein the liquid culture medium is supplemented with a plant oil selected from the group consisting of artichoke oil, canola oil, coconut oil, corn oil, cottonseed oil, mustard oil, olive oil, palm oil, peanut oil, rapeseed oil, safflower oil, soybean oil, sunflower oil, and combinations thereof.

14. The process of claim 10, wherein the plant oil is provided at a concentration from the range of about 0.5% (w/w) to about 30% (w/w).

15. The process of claim 10, wherein the plant oil is provided at a concentration from the range of about 1% (w/w) to about 15% (w/w).

16. The process of claim 1, wherein the liquid culture medium is supplemented with a saturated fatty acid selected from the group consisting of octanoic acid, decanoic acid, dodecanoic acid, tetradecanoic acid, salts thereof, esters thereof, and combinations thereof.

17. The process of claim 10, wherein the saturated fatty acid is provided at a concentration from the range of about 0.05 mM to about 70 mM.

18. The process of claim 10, wherein the saturated fatty acid is provided at a concentration from the range of about 5.0 mM to about 25.0 mM.

19. The process of claim 1, wherein the liquid culture medium is supplemented with a salicylic acid.

20. The process of claim 10, wherein the salicylic acid is provided at a concentration from the range of about 0.5 mM to about 7.5 mM.

21. The process of claim 1, wherein the culture is a combination of at least two Pseudomonas putida strains selected from the group consisting of IDAC 181110-01, IDAC 181110-02, IDAC 181110-03, IDAC 171111-01, and IDAC 171111-02.

22. The process of claim 1, wherein the liquid culture medium is an agri-industrial wastestream selected from the group consisting of cellulose fermentation products generated by cellulytic bacteria, thin stillage recovered from distillation of ethanol in grain ethanol production systems, slurries recovered from distillation of ethanol in grain ethanol production systems, slurries recovered from distillation of ethanol in grain ethanol production systems and amended with dried distiller grains with solubles, slurries recovered from distillation of ethanol in grain ethanol production systems amended with wet cake, thin stillage recovered from distillation of ethanol in cellulosic ethanol production systems, slurries recovered from distillation of ethanol in cellulosic ethanol production systems, hydrolysates produced by enzymatic digestion of hemicelluloses, and hydrolysates produced by chemical digestion of hemicelluloses.

23. A microbial inoculum composition for producing medium-chain-length 3-hydroxyalkanoic acids in a liquid medium, the microbial inoculum composition comprising a culture of a Pseudomonas putida strain selected from the group consisting of, IDAC 181110-01, IDAC 181110-02, IDAC 181110-03, IDAC 171111-01, and IDAC 171111-02.

24. The microbial inoculum composition of claim 23, wherein the culture is a combination of at least two Pseudomonas putida strains selected from the group consisting of IDAC 181110-01, IDAC 181110-02, IDAC 181110-03, IDAC 171111-01, and IDAC 171111-02.

25. A pure culture of Pseudomonas putida IDAC 181110-01.

26. A pure culture of Pseudomonas putida IDAC 181110-02.

27. A pure culture of Pseudomonas sp. K08 IDAC 181110-03.

28. A pure culture of Pseudomonas putida IDAC 171111-01.

29. A pure culture of Pseudomonas putida IDAC 171111-02.

30. A pure culture comprising transgenic microbial cells prepared from a medium-chain-length 3-hydroxyalkanoic acid-producing microbial cell wherein a portion of a phaC1 gene has been deleted.

31. A pure culture comprising transgenic microbial cells prepared from a medium-chain-length 3-hydroxyalkanoic acid-producing microbial cell wherein a portion of a phaC2 gene has been deleted.

32. A pure culture comprising transgenic microbial cells prepared from a medium-chain-length 3-hydroxyalkanoic acid-producing microbial cell wherein a portion of a phaZ gene has been deleted.
